(19) United States
(12) Patent Application Publication
(10) Pub. No.: US 2022/0409855 A1
(43) Pub. Date: Dec. 29, 2022

(54) METHODS OF DELIVERING CELLS AND THERAPEUTIC AGENTS TO ORGANS AND EXTRAVASCULAR SITES

(71) Applicants: Staffan HOLMIN, Bromma (SE); Nevin WITMAN, Portsmouth, NH (US); Jonathan Siradj AL-SAADI, Stockholm (SE); Kenneth CHIEN, Cambridge, MA (US); Mathias WALDÉN, Lidingö (SE); Jesper SOHLMÉR, Solna (SE)

(72) Inventors: Staffan HOLMIN, Bromma (SE); Nevin WITMAN, Portsmouth, NH (US); Jonathan Siradj AL-SAADI, Stockholm (SE); Kenneth CHIEN, Cambridge, MA (US); Mathias WALDÉN, Lidingö (SE); Jesper SOHLMÉR, Solna (SE)

(21) Appl. No.: 17/852,177

(22) Filed: Jun. 28, 2022

Related U.S. Application Data
(60) Provisional application No. 63/216,348, filed on Jun. 29, 2021.

(51) Int. Cl. A61M 25/00

(52) U.S. Cl. CPC A61M 25/0084; A61M 25/0069; A61M 2025/0089

(57) ABSTRACT

Methods of delivering cells and other therapeutic agents to organs and extravascular sites using an endoluminal delivery device are provided that allow for retention of the cells or agents at the delivery site at significantly improved levels as compared to needle delivery. Optimal delivery rates using the endoluminal delivery device are also provided. The methods allow for delivery of a variety of therapeutic agents to organs including heart, kidney and pancreas.

### RELATED APPLICATIONS

This application claims priority to U.S. Provisional Application No. 63/216,348, filed Jun. 29, 2021, the entire contents of which is hereby incorporated by reference.

### BACKGROUND OF THE INVENTION

Cardiovascular disease is one of the leading causes of death worldwide. Moreover, cardiac muscle damage resulting from myocardial infarction can significantly impair cardiac function, ultimately leading to heart failure. Cell transfer therapies, such as with stem cell-derived cardiac progenitor cells, offer potential treatment to restore and improve cardiac function. Additional intracardiac therapies, such as with protein-based or oligonucleotide-based agents, also hold promise for upregulating cardiac activity. Delivery of such therapies to the heart, however, remains challenging.

Various methods for transferring cells to a damaged myocardial site have been investigated, including intravenous infusion (peripheral infusion), coronary sinus infusion, intracoronary infusion, and direct intramyocardial injection using an intracardiac catheter (transendocardial) or through open chest surgery (transepicardial). Transepicardial injections are accomplished by means of thoracotomy, which is not only highly invasive but can also lead to post-operative complications. Consequently, significantly less invasive approaches are favored for intramyocardial delivery. For example, needle-based injection catheter systems have been described for transendocardial delivery.

Such needle-based catheter systems typically use a 26-gauge or 27-gauge-sized needle for injection of cells or other therapeutic agents into the heart. For example, Garbayo et al. describe the use of a 27 gauge NOGA MYOSTAR injection catheter to deliver microparticles loaded with protein therapeutics (FGF₁ or NRG1) by intramyocardial injection into porcine hearts (Garbayo et al. (2016) Sci. Rep. 6:25932). Alternatively, the BioCardia Helical Infusion System uses a helical needle tip, rather than a straight needle tip, for intramyocardial delivery of agents. Additional needle-based catheter systems are reviewed in Cheng, W. and Law, P. K. (2017) Cell Transplantation 26:735-751. Needle-based catheter systems, however, may have limitations, both due to low retention of the delivered cells or agents at the treatment site and possible damage to surrounding cardiac tissue during delivery.

Thus, while various approaches for intraorgan or extravascular delivery of cells and other therapeutic agents have been described, there still exists a need in the art for additional methods and approaches, in particular ones that achieve high retention of the delivered therapeutic with minimal invasiveness.

### SUMMARY OF THE INVENTION

The methods and compositions of the disclosure provide minimally-invasive means for delivering therapeutic agents to intraorgan or extravascular sites that offer significantly improved retention of the therapeutic in vivo as compared to needle-based delivery systems, as well as the ability to repeatedly dose at the intraorgan or extravascular site without harmful effects. In particular, conditions and protocols have been determined for effective delivery of cells and other therapeutics to intraorgan and extravascular sites using an endoluminal delivery cannula. Stem cells and stem cell-derived progenitor cells have been shown to pass through this cannula with a high yield of cell recovery and cell viability (see Examples 1 and 2). Moreover, a dose of cells can be effectively passed through the cannula with high recovery and viability at a rapid rate (passage speed) of approximately 30 seconds to 4 minutes, more preferably 2 minutes or less. In an embodiment, the method comprises delivering cells at a cell concentration of approximately 100,000-200,000 cells/μl (e.g., 133,000 cells/μl) at a passage rate of 60 seconds (see Example 2). In embodiments, the flow rate is 20-2000 μl/minute (e.g., 200 μI/minute). Delivery of labeled stem cells to swine heart in vivo using the cannula led to significantly higher retention of the cells as compared to labeled stem cells delivered using a 26-gauge needle (see Example 3). The level of retention of the therapeutic using cannula-based delivery was at least 2-fold higher, and as much as 9-fold higher, than the level using needle-based delivery.

Accordingly, in one aspect, the disclosure pertains to a method of delivering a therapeutic to an intraorgan or extravascular site in a subject, the method comprising:

(a) providing an endoluminal delivery cannula comprising a dose of the therapeutic; and

(b) administering the dose of the therapeutic to the intraorgan or extravascular site in the subject using the endoluminal delivery cannula,

wherein the dose is administered in 4 minutes or less; and

wherein the dose is retained at the intraorgan or extravascular site at a level at least 2-fold higher than an equivalent dose administered using a 26- or 27-gauge needle.

In various embodiments, the dose is administered in, for example, 3 minutes or less, 2 minutes or less, 90 seconds or less, or 60 seconds or less. In various embodiments, the flow rate is 20-2000 μl/minute, 20-1000 μl/minute, 20-500 μl/minute, 50-1000 μl/minute, 50-500 μl/minute, 100-1000 μl/minute, 100-500 μl/minute, 100-300 μl/minute, 50-200 μl/minute, 20 μl/minute, 50 μl/minute, 100 μl/minute, 150 μl/minute or 200 μl/minute.

In various embodiments, the therapeutic comprises cells at a concentration of 10,000-1,000,000 cells/μl, 10,000-500,000 cells/μl, 10,000-250,000 cells/μl, 25,000-500,000 cells/μ1, 25,000-250,000 cells/μl, 33,000-200,000 cells/μl, 50,000-200,000 cells/μl, 10,000 cells/μ1, 25,000 cells/μl, 33,000 cells/μl, 50,000 cells/μl, 66,000 cells/μl, 100,000 cells/μl, 133,000 cells/μl, 150,000 cells/μl, 200,000 cells/μl, 250,000 cells/μl, 300,000 cells/μl, 350,000 cells/μ1, 400,000 cells/μl, 450,000 cells/μ1, or 500,000 cells/μl.

In various embodiments, the dose is retained at the intraorgan or extravascular site at a level at least 2-fold, 3-fold, 4-fold, 5-fold, 6-fold, 7-fold, 8-fold or 9-fold higher than an equivalent dose administered using a 26- or 27-gauge needle.

In an embodiment, the therapeutic comprises cells, such as stem cells or stem cell-derived progenitor cells. In an embodiment, the therapeutic comprises mesenchymal stem cells (MSCs). In an embodiment, the MSCs are modified to express at least one therapeutic agent. In an embodiment, the MSCs are modified with mRNA encoding the therapeutic agent(s). In an embodiment, the therapeutic comprises human ventricular progenitor cells (HVPs) (e.g., for delivery into the heart, such as intramyocardially). In an embodiment, the therapeutic comprises cells at a concentration in the dose of 100,000-300,000 cells/μl, e.g., the cell concentration in the dose can be 133,000 cells/μl. Various types of cells suitable as therapeutics, as well as doses, are described further herein.

In embodiments, the therapeutic is a protein-based agent. Non-limiting examples of protein-based agents include growth factors, cytokines, chemokines, antibodies, nanobodies, and the like. In embodiments, the therapeutic is an oligonucleotide-based agent. Non-limiting examples of oligonucleotide-based agents include DNA agents and RNA agents, including mRNA agents. Various types of protein-based and oligonucleotide-based therapeutics are described further herein.

In embodiments, the method is used to deliver a therapeutic to an intraorgan site within the heart. In embodiments, the method is used to deliver a therapeutic to an intraorgan site within the kidney. In embodiments, the method is used to deliver a therapeutic to an intraorgan site within the pancreas. In embodiments, the method is used to deliver a therapeutic to an intraorgan site within the liver In embodiments, the method is used to deliver a therapeutic to an intraorgan site within the brain. In embodiments, the method is used to deliver a therapeutic to an intraorgan site within the lung. In embodiments, the method is used to deliver a therapeutic to an an extravascular site of the heart. In embodiments, the method is used to deliver a therapeutic to an an extravascular site of the kidney. In embodiments, the method is used to deliver a therapeutic to an an extravascular site of the pancreas. In embodiments, the method is used to deliver a therapeutic to an an extravascular site of the liver. In embodiments, the method is used to deliver a therapeutic to an an extravascular site of the brain. In embodiments, the method is used to deliver a therapeutic to an an extravascular site of the lung.

Various embodiments of the endoluminal delivery cannula are described in detail herein and illustrated schematically in the drawings. In embodiments, the endoluminal delivery cannula comprises:

an elongated proximal portion having an outer diameter (OD),

a tip portion arranged distally of the proximal portion and extending from the proximal portion to a distal tip of the cannula, and

a lumen having an inner diameter (ID) and continuously extending through the proximal portion and the tip portion to the distal tip, the therapeutic being contained within the lumen; wherein

the tip portion has an opening at the distal tip to provide communication between the continuous lumen and the exterior of the cannula; and

the tip portion is tapered towards the distal tip.

In embodiments, the OD of the elongated proximal portion of the cannula is smaller than the outer diameter of a 26-gauge or 27-gauge needle. In embodiments, the OD of the proximal portion is 200-300 micrometers. In embodiments, the OD of the proximal portion is 250 micrometers.

In embodiments, the inner diameter (ID) of the lumen of the endoluminal delivery cannula is smaller than the inner diameter of a 26-gauge or 27-gauge needle. In embodiments, the lumen of the cannula has an ID of 140-160 micrometers. In embodiments, the lumen has an ID of 145-150 micrometers.

In embodiments, the tip portion has a longitudinal length (L₃) within a range of 5 mm to 300 mm. In embodiments, the longitudinal length (L₃) of the tip portion is less than 100 mm. In embodiments, the longitudinal length L₃ of the tip portion is within the range of 5 mm to 10 mm, 5mm to 15mm, 5 mm to 20 mm, 5 mm to 25 mm, 5 mm to 30 mm, 5 mm to 35 mm, 5 mm to 40 mm, 5 mm to 45 mm or 5 mm to 50 mm.

In embodiments, the tip portion has a proximal end with an outer diameter (D₃) and a distal end at the distal tip with an outer diameter (D₄), wherein the outer diameter (D₃) is the same as the OD of the elongated proximal portion and the outer diameter (D₄) is smaller than the outer diameter (D₃). In embodiments, the outer diameter (D₃) is 225-275 micrometers and the outer diameter (D₄) is 175-200 micrometers. In embodiments, the outer diameter (D₃) is 250 micrometers and the outer diameter (D₄) is 194 micrometers.

In embodiments, the tip portion is non-helical.

In embodiments, the dose is administered to the intraorgan or extravascular site in the subject by:

a) navigating the distal tip of the cannula to a location near the intraorgan or extravascular site in the subject;

b) directing the distal tip toward a vessel wall in a general direction of the intraorgan or extravascular site;

c) advancing the distal tip such that the distal tip penetrates the vessel wall and reaches the intraorgan or extravascular site;

d) injecting the dose into the intraorgan or extravascular site; and

e) retracting the distal tip of the cannula from the intraorgan or extravascular site of the subject.

In embodiments, more than one dose of the therapeutic is administered to the subject by repeating the injection at the intraorgan or extravascular site using one or more additional doses of the therapeutic. Accordingly, the method can comprise repeating steps a) through e) with a second dose of the therapeutic.

These and other aspects of the disclosure are described in further detail herein.

### BRIEF DESCRIPTION OF THE DRAWINGS

FIG. 1 shows an overview of use of an endoluminal delivery assembly as disclosed herein.

FIGS. 2A and 2B illustrate a first aspect of an endoluminal delivery cannula.

FIGS. 3A and 3B illustrate further aspects of an endoluminal delivery cannula.

FIGS. 4A and 4B illustrate a cross-sectional view of a cannula hub.

FIGS. 5A and 5B show perspective and side views, respectively, of an assembly comprising an endoluminal delivery cannula.

FIGS. 6A and 6B illustrate cross-sectional views of specific parts in an assembly comprising an endoluminal delivery cannula.

FIGS. 7A-7E illustrate reference planes and needle grinding angles for a cannula tip section.

FIG. 8 shows a perspective view, a top view and a side view of a preferred cannula tip section.

FIG. 9 shows bar graphs showing VEGF production by electroporated mesenchymal stem cells (MSC EL), cryopreserved MSCs after thawing (MSC cryo), or MSCs passaged through the Extroducer (Cath1 and Cath2) after 24 hours, 48 hours or 72 hours.

FIG. 10 shows a bar graph showing VEGF production in swine hearts treated using the Extroducer with either VEGF-encoding mRNA in a citrate saline formulation (“Naked”), unmodified mesenchymal stem cells (MSCs)(“Naive treatment”) or MSCs loaded with VEGF-encoding mRNA (“Enhanced”).

FIGS. 11A-11C are photographs of histology slides from the kidney cortex of swine following injection of MSCs, using the Extroducer, followed by methylene blue injection as a label for the injection site. To visualize the human MSCs, kidney sections were stained with anti-human nuclear antigen antibody (anti-NA Ab). FIG. 11A shows human cells labeled with anti-NA Ab. FIG. 11B shows all cells labeled with DAPI. FIG. 11C shows cells labeled with anti-NA Ab and DAPI.

FIGS. 12A-12C are photographs of histology slides from the kidney medulla of swine following injection of MSCs, using the Extroducer, followed by methylene blue injection as a label for the injection site. To visualize the human MSCs, kidney sections were stained with anti-human nuclear antigen antibody (anti-NA Ab). FIG. 12A shows human cells labeled with anti-NA Ab. FIG. 12B shows all cells labeled with DAPI. FIG. 12C shows cells labeled with anti-NA Ab and DAPI.

### DETAILED DESCRIPTION OF THE INVENTION

The methods and compositions of the disclosure pertain to delivery of cells and/or other therapeutic agents into an organ of a subject or into an extravascular site of the subject such that the therapeutic is retained more effectively than with needle-based delivery systems. The disclosure is based, at least in part, on the discovery of conditions and protocols (e.g., as described further in the Examples) that allow for high recovery and viability of the therapeutic and significant in vivo retention using an endoluminal delivery cannula. The methods and compositions of the disclosure allow for delivery of therapeutics to organs such as heart, kidney and pancreas, useful in the treatment of a wide variety of diseases and disorders.

### I. Endoluminal Delivery Cannula

As used herein, the terms “endoluminal delivery cannula”, “endoluminal delivery catheter”, “endoluminal delivery device”, “Extroducer” and “trans-vessel wall device” (trans-VW device) are used interchangeably to refer to the device described herein for delivery of therapeutics to an intraorgan or extravascular site.

Endoluminal delivery cannulas are generally described in, for example, PCT Publication WO 2009/124990, PCT Publication WO 2012/004165, EP Patent 2291213B and U.S. Pat. No. 8,876,792, as well as Grankvist et al. (2019) J. Int. Med. 285:398-406, the contents of each of which is hereby specifically incorporated by reference.

Specific embodiments of the endoluminal delivery cannula will now be described. However, it will be apparent to those skilled in the art that individual features may be combined in different manners, and the below disclosures are in no manner limiting.

The terms “proximal” and “distal” is herein used as is conventional in the art, i.e. as being in relation to a user, such that a proximal end of a device or assembly is the end directed towards the user, and a distal end is directed away from a user.

Further, the terms “cannula” and “needle” are used interchangeably herein, and both refer to an elongated tube, preferably made of a metal, which may have a sharpened tip adapted for penetration of tissue or the like.

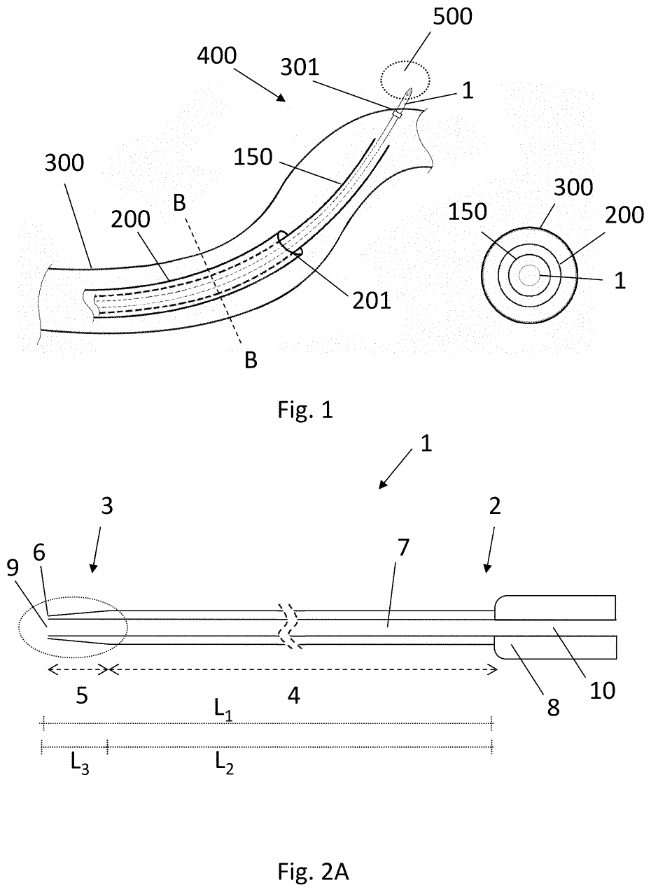

FIG. 1 shows an overview of an endoluminal delivery cannula 1 as disclosed herein in use, when arranged within a blood vessel 300. The cannula 1 may preferably be provided in a kit or assembly 400 together with a protective catheter 150, and adapted for insertion into the vascular system of a human or animal body via a guide catheter, which is described in more detail further below.

To initiate a delivery procedure, a guide catheter 200 is typically used to reach a location near a defined target site 500 via the vascular system. Such a guide catheter 200 is typically a standard interventional catheter for vascular access, and may be provided together with the disclosed cannula 1 and protective catheter 150, or as a separate unit. On the right side in FIG. 1, a cross-sectional view through the plane B is illustrated schematically, wherein it is seen that the protective catheter 150 is preferably coaxially arranged around the cannula 1, and the guide catheter 200 is arranged around the protective catheter 150 within the vessel 300.

The guide catheter 200 may be inserted percutaneously into a blood vessel 300 according to known techniques, e.g. using the Seldinger procedure or other known techniques, to access the vasculature via for instance the femoral artery or the radial artery. It should be noted that the devices and assemblies described herein are especially adapted for access to remote target sites in the body, i.e. for access into and via the microvasculature, and thus adapted to navigate very small vessels, as small as 1 mm or less in diameter, to reach sites within the body previously not accessible via standard techniques. However, they are also compatible for use with larger guide catheters and via larger blood vessels. The devices, assemblies and methods described herein are described in terms of reaching an extravascular target site; however, they may also similarly be used for intramyocardial delivery. Thus, a target site may be accessed via navigation through the vascular system and reached either via penetration of a blood vessel wall (for an extravascular site) or via penetration of the myocardium from e.g. inside the heart (for an intramyocardial target site).

The disclosed devices and assemblies may be used for targeted and localized delivery of one or a combination of cells, RNA, recombinant proteins, antibodies, high-dose chemotherapy, radiotherapy, or tumor-specific therapies. The specific target site 500 may be a tumor, an organ, a body cavity or a localized region of a specific tissue or body part. As further examples, disclosed devices and assemblies may be used for delivery of e.g. cell or RNA therapy for cardiometabolic regenerative therapies including the heart, liver, pancreas, and kidney, for neuroregenerative therapies including the brain and for direct intra-tumor infusion.

When the guide catheter 200, which preferably has maneuverability and steerability properties, has been inserted via the vasculature such that the distal tip 201 of the guide catheter 200 is near the desired target site 500, the cannula 1 and protective catheter 150 are inserted via the guide catheter 200. As an alternative, the cannula 1 may be inserted into the guide catheter prior to inserting the assembly into the vasculature. In any case, the cannula 1 and protective catheter 150 are adapted to be navigated through the vascular system of a patient to a location near a target site 500 via the guide catheter 200. The cannula 1 and protective catheter 150 are directed towards the vessel wall 301, as seen in FIG. 1. In some aspects, not shown in FIG. 1, a guide catheter 200 with a pre-shapeable and pre-bent tip 201 may be used, such that when the tip is near the target site, the bent tip will further assist in guiding the assembly towards the vessel wall 301. Alternatively or in combination, a steerable tip 201 is also envisioned to assist in fine adjustments of the injection direction.

During the insertion through the guide catheter 200 and out towards the vessel wall 301, the protective catheter 150 and cannula 1 are prevented from axial displacement in relation to each other by a locking function at the proximal end, which will be described further below. This is to prevent the potentially sharp tip of the cannula 1 from piercing or damaging the guide catheter or the blood vessel before reaching the desired location.

Once at the desired site at the vessel wall 301, the tip of the cannula 1 is advanced out of the protective catheter 150 towards the vessel wall and further distally, such that it penetrates the vessel wall and extravascular tissue to reach the target site 500, as shown in FIG. 1. In some aspects, the tip of the cannula 1 may be provided with a depth limit element, as will be described further below. The advancement of the tip may preferably also be viewed by providing one or several radiopaque markers near or at the tip, such that the tip may be located during the procedure using angiography or other imaging techniques.

Once at the target site 500, a substance to be injected into the target site is applied by syringe into the cannula via a proximal cannula hub, and ejected from the cannula via a distal opening at the tip of the cannula. In some aspects, the injected substance may be ejected both via the distal opening, and via side openings near the distal tip of the cannula. The administration of substance may be repeated a number of times, as needed. Further, the tip of the cannula may be retracted and repositioned if needed. Once administration is finished, the tip of the cannula is retracted from the vessel wall and back into the protective catheter.

As an alternative, or in addition to administrating a substance, the assembly may be used for taking samples from the target site via the tip of the cannula.

Due to a specific design of the cannula and its tip, as well as the OD dimension (which is significantly smaller than a 26- or 27-gauge needle), essentially no bleeding of the vessel wall is seen. The details of the tip will described further below.

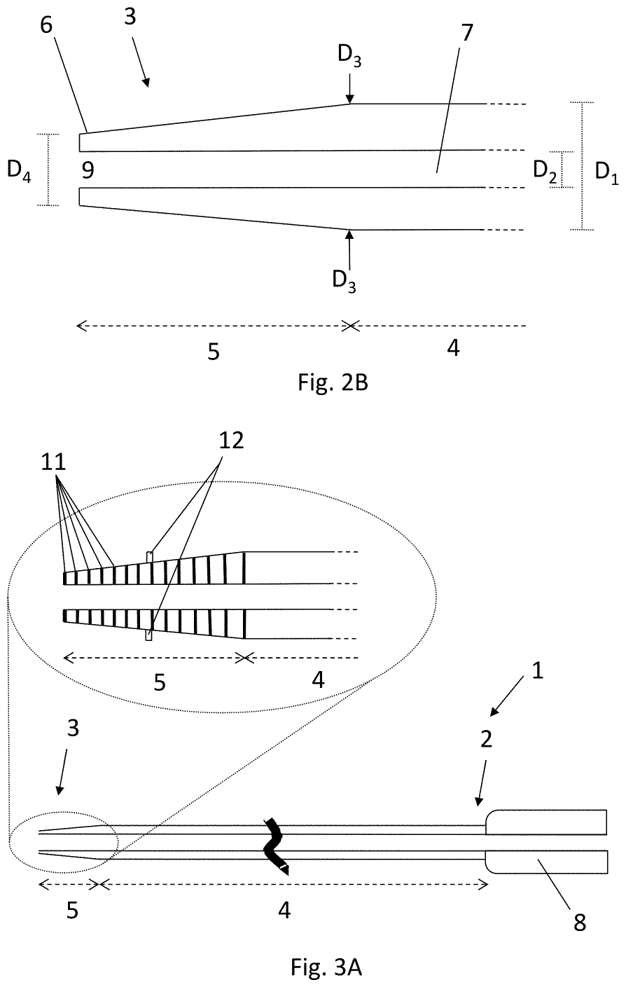

FIG. 2A schematically illustrates a cross-sectional view along a longitudinal axis of the endoluminal delivery cannula 1 as disclosed herein. As described above, the cannula 1 is adapted for delivery of a substance to an extravascular or intraorgan (e.g., intramyocardial) target site via the vascular system of a human or animal body. The cannula 1 has a proximal end 2 configured to remain outside the body and a distal end 3 configured to be inserted into the body via the vascular system to access an extravascular target site. A cannula hub 8 is preferably provided at the proximal end 2 of the cannula 1. The majority of the longitudinal length L₁ of the cannula is made up of an elongated proximal portion 4, which has a constant outer diameter D₁ along essentially the entire length of the proximal portion 4. Notably, a “constant diameter” herein means that the diameter is constant as measured when the cannula is in an elongated or essentially straight configuration, as bending may cause distortions to the diameter.

The cannula 1 further has a tip portion 5 arranged distally of the proximal portion 4 and extending from the proximal portion 4 to a distal tip 6 of the cannula 1. A continuous lumen 7 extends from an internal longitudinal channel 10 of the cannula hub at the proximal end 2 of the cannula through the proximal portion 4 and the tip portion 5 to the distal tip 6. The tip portion 5 has an opening 9 at the distal tip 6 to provide communication between the lumen 7 and the exterior of the cannula 1. In embodiments, the tip portion is non-helical.

The encircled tip portion 5 in FIG. 2A is shown in an enlarged and more detailed illustration in FIG. 2B. The tip portion 5 is tapered towards the distal tip 6, and thus has an outer diameter D₃ at the proximal end of the tip portion 5, and another smaller outer diameter D₄ at the distal tip 6. The outer diameter D₃, is essentially the same as the outer diameter D₁ of the elongated proximal portion 4, and the outer diameter D₄ is smaller than the outer diameter D₃. In other words, a gradual taper is provided in a distal direction from the point where the proximal portion 4 transitions into the distal tip portion 5 to the distal end 6.

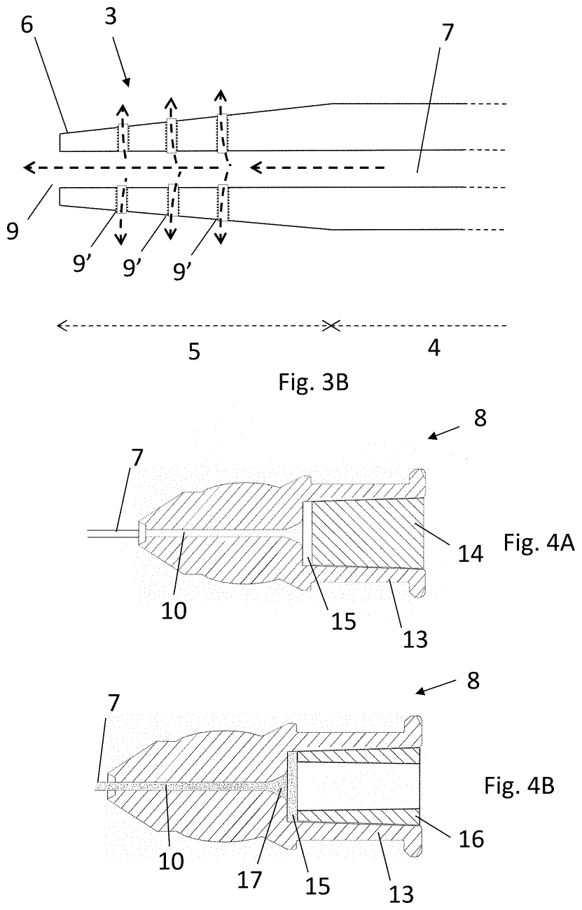

In some aspects, the tip portion 5 and/or proximal portion 4 may preferably be provided with one or several radiopaque marker bands 11 at predefined distances from the distal end 6. One such example is schematically shown in FIG. 3A, illustrating several radiopaque marker bands 11. Such marker bands are clearly visible using angiographic imaging during a procedure, and thus used to determine the exact location of the tip and the penetration depth into a target site. FIG. 3A shows only one schematic example of providing radiopaque marker bands 11. In practice, bands may be provided in any pre-defined configuration, such that they may be used for localization of the tip, and, in particular, to guide the user and determine the penetration depth from the vessel wall to the target site.

Further, in some aspects, also shown in FIG. 3A, the tip portion 5 may be provided with one or several outwardly protruding depth limit elements 12, e.g. in the form of circumferential flanges or similar structures, such that a resistance is felt by the user when the depth limit elements 12 reach the vessel wall on penetration. In one aspect, a depth limit element may coincide with the transition from the tip portion 5 and the proximal portion 4. A cannula 1 may have either radiopaque markers 11 or depth limit elements 12, or both.

As is understood from the figures, and from the present disclosure, opening 9 at the distal tip 6 provides communication between the lumen 7 and the exterior of the cannula 1 and thus, when the substance to be delivered exits the cannula into the target site, the substance exits the cannula through this distal opening 9. Substance delivery is thus controlled and easily directed in the direction that the cannula is directed.

In some aspects, as illustrated in FIG. 3B, the cannula may be provided with one or several secondary side openings 9′, near the distal opening 9, and thus distal to any radiopaque markers 11 and/or depth limit elements 12. Such side openings allow the substance to exit the cannula through both the distal opening 9 and the one or more side openings 9′, and provide a wider and/or more rapid distribution of substance at a target site, as is shown by the dashed arrows in FIG. 3B. Side openings 9′ allow ejection from the cannula in a radial direction, in addition to the distal direction, provided by the distal opening 9. Depending on the intended distribution, side openings 9′ may be adapted to a specific pattern and/or opening size. In one aspect, one or several side openings 9′ are provided along a circumference of the tip portion 5. The side openings 9′ may be provided in a random pattern or a predefined pattern. In some aspects, side openings 9′ are arranged near the distal opening 9. Notably, the aspects described in correlation to FIGS. 3A and 3B may thus be combined.

As detailed in FIG. 2A, and applicable for all cannulas disclosed herein, the cannula 1 may have a total longitudinal length L₁ from the proximal end 2 to the distal end 3 within in the range of approximately 300 mm to 2500 mm. In some aspects, mainly for use in adult patients, the total longitudinal length L₁ is preferably between 1200 mm to 1900 mm, or more preferably 1650 mm to 1750 mm. However, for e.g. pediatric use a total longitudinal length L₁ of approximately 300 mm to 800 mm is preferred.

The longitudinal length L₁ of the proximal portion 4 may be within in the range of approximately 1000 mm to 2000 mm, preferably between 1200 mm to 1700, more preferably 1400 mm to 1500 mm.

In some aspects, the tapered tip portion 5 has a longitudinal length L₃ of at least 5 mm, preferably within in the range of 100 mm and 300 mm, more preferably between 200 mm and 280 mm. In some aspects the longitudinal length L₃ of the tapered tip portion 5 may be within the range of 5 mm to 50 mm, in other aspects the longitudinal length L₃ may be within the range of 50 mm to 300 mm. In some aspects the longitudinal length L₃ of the tapered tip portion 5 may be within the range of 5 mm to 10 mm, 5mm to 15mm, 5 mm to 20 mm, 5 mm to 25 mm, 5 mm to 30 mm, 5 mm to 35 mm, 5 mm to 40 mm, 5 mm to 45 mm or 5 mm to 50 mm.

In one aspect, the total length L₁ of the cannula 1 is approximately 1700 mm, wherein the proximal portion 4 has a longitudinal length L₁ of approximately 1450 mm and the tip portion 5 has a longitudinal length L₃ of approximately 250 mm.

In another aspect, the total length L₁ of the cannula 1 is approximately 1700 mm, wherein the proximal portion 4 has a longitudinal length L₁ of approximately 1695 mm and the tip portion 5 has a longitudinal length L₃ of approximately 5 mm. Such a cannula would thus have essentially no or minimal tapered portion.

In yet another aspect, the total length L₁ of the cannula 1 is approximately 500 mm, wherein the proximal portion 4 has a longitudinal length L₁ of approximately 425 mm and the tip portion 5 has a longitudinal length L₃ of approximately 75 mm. Such a size would be useful for pediatric use.

The proximal portion 4 of the cannula 1 preferably has a constant outer diameter D₁ within the range of 0.15 mm to 0.50 mm, preferably between 0.20 mm and 0.35 mm, more preferably between 0.25 mm to 0.28 mm.

The inner lumen 7 preferably has an inner diameter D₂ which is constant along essentially the entire length of the cannula 1, i.e. through the proximal portion 4 and the tip portion 5. Naturally, the inner diameter D₂ must be adapted to a suitable outer diameter D₁ of the cannula. The inner diameter D₂ of the lumen is preferably within in the range of approximately 0.08 mm to 0.40 mm, preferably between 0.10 mm and 0.25 mm, more preferably between 0.12 mm and 0.16 mm.

As mentioned, the outer diameter D₁ of the proximal portion 4 is preferably essentially constant along essentially the entire length of the proximal portion 4. Further, the part of the cannula where the proximal portion 4 adjoins the tip portion 5 has an outer diameter D₃ being essentially the same as the outer diameter D₁ of the elongated proximal portion 4. In other words, the cannula preferably has a smooth transition in outer diameter from the proximal portion 4 to the tip portion 5. Thereafter, the tip forms a gradual tapered tip towards the distal end 6, such that the outer diameter D₄ at the distal end 6 is smaller than the outer diameters D₃ and D₁. This taper is preferably provided such that the outer diameter D₄ at the distal tip 6 is preferably between 0.10 mm and 0.25 mm, and more preferably between 0.15 mm to 0.22 mm.

In one aspect, the outer diameter D₁ of the proximal portion 4 may be approximately 0.25 mm, the inner diameter D₂ of the lumen 7 approximately 0.134 mm and the outer diameter D₄ at the distal end 6 is 0.190 mm.

The gradual tapered tip portion 5 provides improved maneuverability, trackability and mainly pushability of the cannula tip, as well as providing a gradual transition to a smaller size distal tip. A smaller size tip inflicts less trauma on the vessel wall during penetration, and the small diameter of the tip allows the vessel wall to close in on itself after withdrawal of the tip, such that less bleeding is experienced after delivery. Thus, the configuration of the tip portion mitigates the need for any separate closure steps of the penetration site of the vessel wall.

The elongated proximal portion 4 and tip portion 5 of cannula 1 may preferably be made of stainless steel, nitinol, or any alloy with superelastic properties, such as Fe—Co—Ni—Ti alloys. In one aspect, the elongated proximal portion 4 and tip portion 5 are made entirely of nitinol or other nickel-titanium alloy. In another aspect, the tip portion 5 may be made of nitinol and the proximal portion 4 made of stainless steel. In further aspects, the tip portion may be made of nitinol with a tip made of a suitable ceramic material. Nitinol's superelastic properties resulting in superior flexibility provides improved navigation through small and tortuous vessels. Having a more rigid distal tip, such as a ceramic tip on a superelastic tip portion, improves the ease of penetration of the distal tip.

As seen in FIGS. 2A and 3A, the cannula 1 is provided with a proximal cannula hub 8. Such a cannula hub 8 is adapted for delivery of a substance, from a syringe or via another adaptor or connector, to the inner lumen 7 of the cannula for further delivery to the target site at the distal end of the cannula 1. The devices and assemblies described herein are especially adapted for delivery of very small volumes of various substances. As mentioned before, substances may include chemotherapy, stem cells, RNA, orphan drugs etc., and such substances are typically very expensive. Thus, achieving a delivery system adapted for minimal loss of volume during delivery has great value. A further advantage of providing a delivery system with minimal dead volume within the system is that the risk of ejecting air bubbles into the target site is minimized, thus mitigating any air embolisms, which could cause a stroke or cardiac arrest, depending on the location within the body.

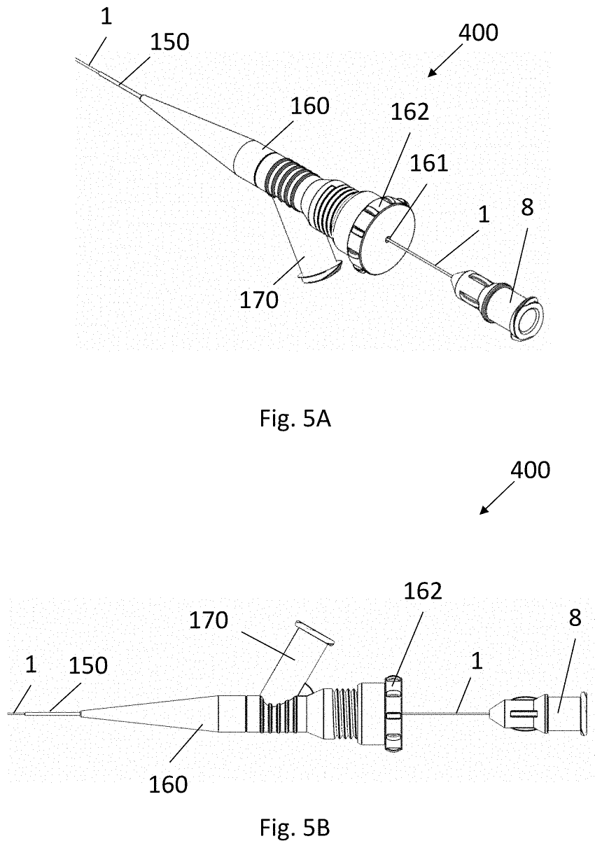

A cross-sectional view along a longitudinal axis of a preferred aspect of a cannula hub 8 is illustrated in FIG. 4A. Notably, FIGS. 4A and 4B are cross-sectional views along a longitudinal axis of an essentially cylindrical or conical cannula hub. In other words, the cannula hub 8 preferably has a round shape being symmetric in all radial directions, as is also seen in FIGS. 5A and 5B, from perspective views. As seen in FIG. 4A, the internal longitudinal channel 10 of the cannula hub 8 connects the lumen 7 of the proximal part 4 of the cannula to the female connector 13 at the proximal end of the cannula hub 8. The female connector 13 may be a standard Luer female connector, or other suitable connector. Before use in delivery of a substance, the cannula hub 8 may be provided with a cap or stopper 14, to keep the internal area clean and contaminant free.

The inner cavity 15 of the cannula hub 8 is preferably adapted for minimal dead volume during delivery when using a standard Luer connector 16, as shown in FIG. 4B. “Dead volume” is herein used as a term for the volume of e.g. a substance which does not exit the device during delivery, e.g. the volume that remains in the cannula hub (or the system as a whole) after a syringe is pressed into the female connector 13 and a substance 17 is injected via the syringe to the cannula. In other words, the inner cavity 15 of the cannula hub 8 is configured such that when a standard male Luer connector 16 is connected to the cannula hub it fits tightly along the inner walls of the cavity 15, and a minimal inner volume is present distally of the male connector 16. The shape of the inner volume may preferably be as illustrated in FIG. 4A and 4B. Thus, the inner volume of the cannula hub 8 may be formed of a thin cylindrical disc distally of the male connector 16, a cone shaped volume and a narrow cylindrical volume of the inner longitudinal channel 10. The dead volume formed by the inner volume of the cannula hub 8 is preferably less than 0.45 ml, preferably within the range of 0.25 ml to 0.45 ml, more preferably between 0.30 ml to 0.40 mm.

The dead volume of the delivery system as a whole comprises the inner volume of the cannula hub 8, as described above, together with the inner volume of the rest of the cannula 1. A small dead volume of the delivery system, as seen when a male Luer connector is attached, allows minimal loss of substance during delivery, which is particularly important when delivering expensive and/or rare substances, and minimizes the risk of creating air embolisms. Thus, preferably the total inner deadspace of the cannula hub 8 and cannula 1 is below 0.50 ml, more preferably below 0.40 ml.

A further advantage of the particular inner volume shape of the cavity of the cannula hub as shown in FIGS. 4A and 4B, i.e. a thin cylindrical disc transitioning smoothly into the inner longitudinal channel 10 via a short cone shape, is that less turbulence is seen in the solution when injecting the substance. This is of particular importance when using delicate substances such as solutions containing living cells, e.g. stem cells, or other less stable cells or molecules.

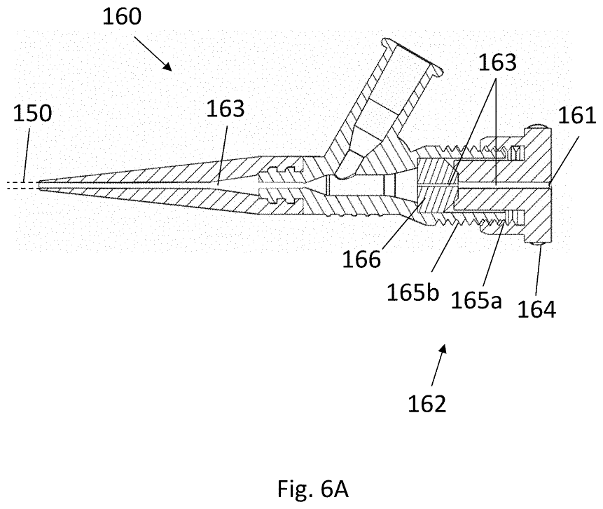

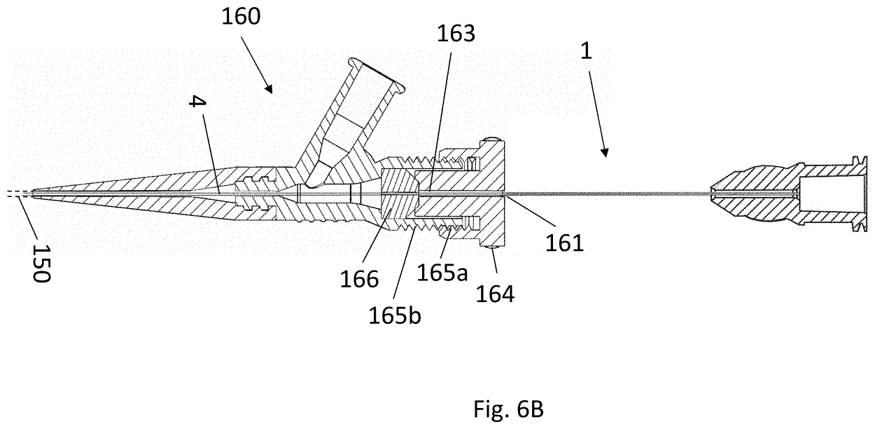

The cannula 1 and cannula hub 8 are preferably used together with a protective catheter 150 and catheter hub 160. One such assembly 400 is illustrated in FIG. 5A, in perspective view, and in FIG. 5B, in a side view. The protective catheter 150 is integrated with or attached to the catheter hub 160 at the distal end of the catheter hub 160. In FIGS. 5A and 5B protective catheter 150 is shown in cut-away view; however, it extends to cover the entire cannula 1 to the distal end of the cannula. FIG. 6A illustrates a cross-sectional view along the longitudinal axis of the catheter hub 160. FIG. 6B illustrates the catheter hub 160 as shown in FIG. 6A with a cannula 1 inserted.

The cannula 1 is adapted to be inserted from the proximal end of the catheter hub 160 through opening 161. A locking means 162 is adapted to be used to lock the cannula 1 in place after insertion of the cannula into the protective catheter 150 and during different stages of the delivery procedure. When the locking means 162 is in a locked state, all axial movement between the protective catheter 150 and the cannula 1, and thus also the cannula hub 8 and the catheter hub 160, is prevented. Before a delivery procedure, the cannula 1 is inserted into the catheter 150 via the catheter hub 160 either by the user, or during manufacture of the assembly, such that the distal tip 6 of the cannula 1 is protected by a distal end of the protective catheter 150 (not illustrated). Notably, in FIGS. 5A and 5B the distal end of the protective catheter is not shown, in order to illustrate that the cannula is located inside the catheter. Further, only a short proximal part of both the cannula and protective catheter 150 are illustrated in the FIGS. 5A and 5B.

As seen in FIGS. 5A and 5B, the catheter hub 160 may comprise a side port 170, which may be used for flushing the system with saline or other suitable solutions, before, during or after the delivery procedure.

As is shown in the cross-sectional view of FIG. 6A, the catheter hub 160 comprises an internal channel 163 which extends from the proximal opening 161 along the longitudinal axis into the protective catheter 150 at the distal end of the catheter hub 160. As is seen in FIG. 6B, the internal channel 163 is adapted for insertion of the cannula 1.

The locking means 162 may comprise any suitable mechanism to be able to lock the cannula in place when inserted into the catheter hub. Preferably, the locking means 162 is adapted to reversibly alternate between a locked state and an unlocked state. Non-limiting examples include screw locks, snap locks, friction locks and lever-based locks. FIGS. 5A-5B and 6A-6B show one example of a locking means 162. At the proximal end of the catheter hub 160, a locking wheel 164 comprising an extension of internal channel 163 is provided. Locking wheel 164 is provided with internal threads 165a, which are adapted to collaborate with external threads 165b on the catheter housing. When the locking wheel 164 is screwed in a distal direction, an internal locking gasket 166 is compressed and the internal channel 163 will be compressed within the gasket 166. Thus, when the cannula 1 is present in the internal channel 163, as is seen in FIG. 6B, it will be gripped by the gasket 166 as the gasket 166 is compressed. Thus, the cannula 1 will be held in place by the gasket 166, thereby hindering any relative axial movement between the catheter hub 160 and cannula 1, and in so doing also any axial movement between the catheter 150 and the cannula 1. By unscrewing the locking wheel 164, the cannula 1 may be released and thereafter repositioned and optionally locked again. Thus, by using locking means 162, the relative axial position of the cannula 1 in relation to the catheter 150 may be adjusted. Further, when locked in relation to each other, the entire assembly may be steered and maneuvered as a single unit.

As described above, before employing the assembly 400 of protective catheter 150 with attached catheter hub 160, and cannula 1 with attached cannula hub 8, a guide catheter is usually placed in a vessel such that the distal end of the guide catheter is as close to the target site as possible. The assembly 400 is inserted into the guide catheter 200 and pushed or guided into a position such that the distal end of the catheter 150 protrudes from the guide catheter 200, as seen in FIG. 1. During this step, the cannula 1 and protective catheter 150 are locked such that axial movement in relation to each other is prevented. This is to inhibit the potentially sharp tip of the cannula 1 from piercing or damaging the guide catheter 200 during insertion, and also to not damage the blood vessel before reaching the desired location. Preferably, the distal end of the assembly 400 is positioned such that the longitudinal axis at the tip of the cannula 1 is directed towards the target site 500, as illustrated. In some aspects, this may be achieved by using a guide catheter 200 with a pre-shaped bent tip and/or a steerable tip 201, as is known in the art.

Once the distal tip 201 is directed towards the vessel wall and the target site 500, the assembly 400 is advanced distally out of the guide catheter 200. At this stage, the distal tip 6 of the cannula 1 is still contained within the protective catheter 150, to avoid any unintended damage to the vessel.

Thereafter locking means 162 of the catheter hub 160 is released, and the tip of the cannula 1 is advanced out of the protective catheter 150 towards the vessel wall and further distally, such that it penetrates the vessel wall and extravascular tissue to reach the target site. This movement is performed by moving the proximal cannula hub 8 closer to the proximal end of the catheter hub 160, by e.g. holding the catheter hub 160 still and moving the cannula hub 8 distally.

In some aspects, a stop element may be provided to prevent premature advancement of the distal tip before reaching a desired location. Such a stop element could be a stop ring or similar arrangement around the cannula 1 between the cannula hub 8 and the catheter hub 160, that may be manually removed before penetration of the vessel wall. As an alternative, or in combination with a stop element, a marker may be provided on the cannula 1 at a location such that it can be seen between the cannula hub 8 and locking means 162 of the catheter hub 160 when the distal tip 6 of the cannula is protected by the distal tip of the protective catheter 150. Such a marker provides a user with a visual indication of when the sharp tip is in a retracted and protected position within the protective catheter, and is useful both during initial positioning of the tip and when repositioning the endoluminal delivery device.

At any desired time during delivery of a substance, such as when the cannula tip protrudes from the catheter tip, the cannula 1 and catheter 150 may be locked in a relative axial arrangement, e.g. by engaging locking means 162. After delivery, or if the cannula tip is to be repositioned, the procedure may be reversed such that the cannula tip is once more protected by the catheter tip, and thereafter optionally repeated for another delivery dose.

As described above, in some aspects the distal tip portion 5 is preferably gradually tapered towards the distal tip 6.

Further, in some aspects, the distal tip portion 5 of the cannula is preferably provided with a pointed tip section 100 for penetrating tissue formed by at least one primary facet F₁ and two secondary facets F₂ and F₃. Notably, herein such a tip section is shown on a cannula 1 for delivery of a substance via the vasculature. However, it is also conceivable to use a similar pointed tip for other devices with similar use, such as micro-needles for intramuscular or intradermal injections.

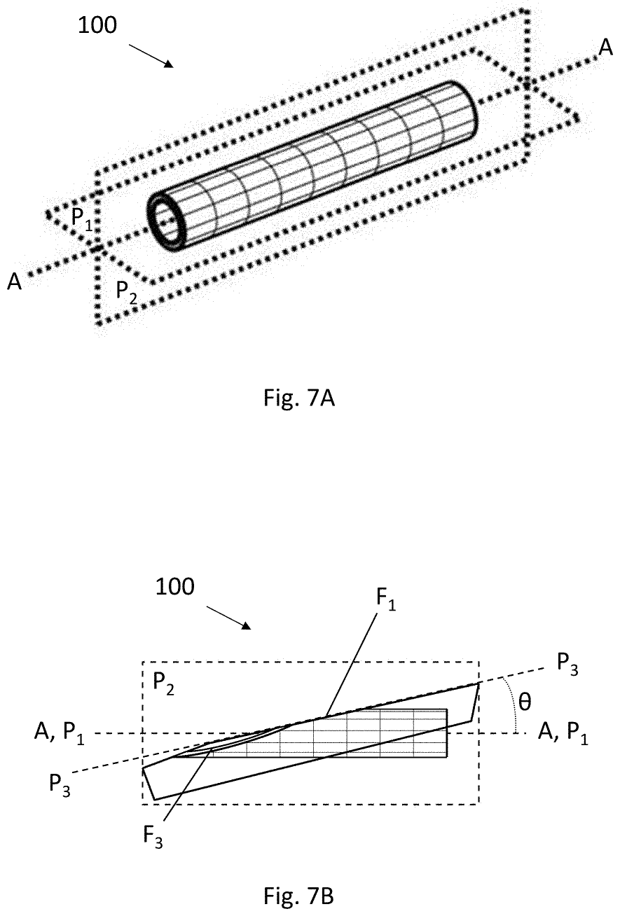

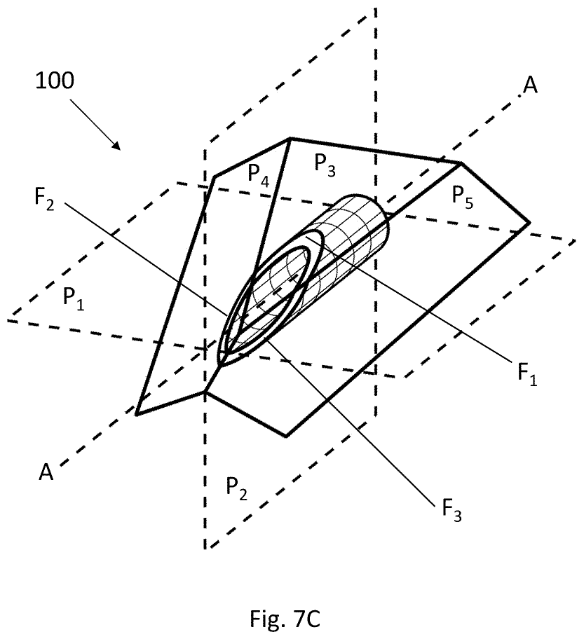

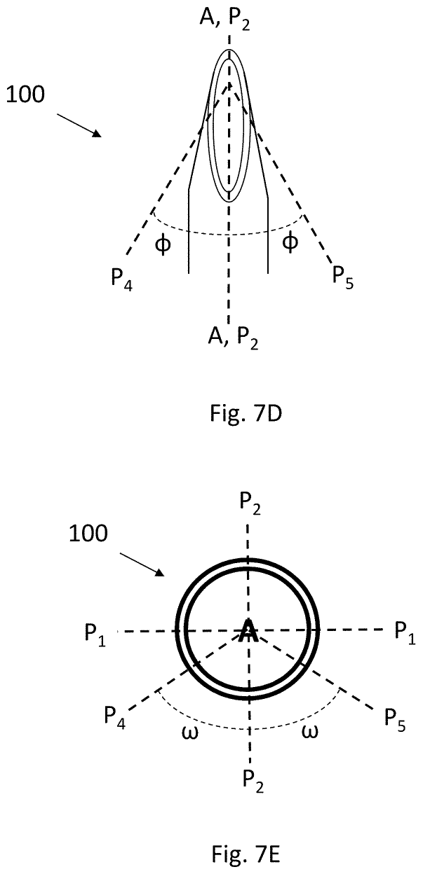

FIGS. 7A-7E illustrate the grinding angles used to form the primary and secondary facets. Notably, the gradual taper of the distal tip portion 5 of the cannula is not visible in FIGS. 7A-7E, and the following figures, as they schematically illustrate only the final approximate 1-3 millimetres of the tip section 100. Furthermore, in FIGS. 7A, 7B, 7C and 8-11A-11C, i.e. when showing a perspective or side view, the left side of the figure is directed generally in the distal direction, and the right side of the figure is directed generally towards the proximal end of the cannula. In FIG. 7D, showing a top view of a tip section 100, upwards in the figure corresponds to a distal direction of the tip. In FIG. 7E, a tip section 100 is viewed along the longitudinal axis A.

In the context of needle grinding, i.e. forming a sharpened tip from a hollow cylindrical cannula, the distal end of the cannula is ground down and sharpened against a grinding wheel or other grinding media. Normally, the grinding wheel is stationary, and the needle or cannula is applied at a fixed angle in relation to the grinding surface. The resulting facets or bevels are thus formed in one or several planes which may be defined in relation to the geometry of the cannula itself.

FIG. 7A shows a perspective and schematic view of a tip section 100 before grinding of any facets. The cylindrical cannula tip section 100 is illustrated to have two reference planes, shown as a first plane P₁ and a second plane P₂ arranged along the longitudinal axis A, wherein the first and second planes P₁, P₂ are perpendicular to each other. These reference planes P₁, P₂ are herein used to define the angles and placement of the planes defining the facets of the resulting tip section 100. For ease of understanding, the first plane P₁ may be viewed as a horizontal plane and the second plane P₂ may be viewed as a vertical plane.

FIG. 7B illustrates a side view of the tip section 100 shown in FIG. 7A after grinding of the facets. A perspective view of the tip section 100 is shown in FIG. 7C, wherein the planes forming the facets in relation to the reference planes are illustrated, as will be detailed below. In a first aspect, the primary facet F₁ of the tip is formed by a needle grinding in a third plane P₃, wherein the third plane P₃ is positioned at an angle theta θ, in respect to the first plane P₁, as best seen in FIG. 7B. Preferably, the third plane P₃ is symmetrically arranged in relation to the second plane P₂ such that the third plane P₃ intersects the first plane P₁, in an extension perpendicular to the second plane P₂. In other words, a pointed tip 6 is formed in the distal direction.

Following the formation of the primary facet F₁, two secondary facets, F₂ and F₃, are formed by a needle grinding of the distal tip in a fourth and fifth plane, P₄ and P₅, respectively, as is illustrated in FIG. 7C. The arrangement of fourth and fifth planes P₄, P₅ is further explained by way of FIG. 7D and 7E.

FIG. 7D illustrates a top view, i.e. as seen from a direction perpendicular to plane P₁, of the tip section 100 after a first grinding in the third plane P₃. FIG. 7E shows the tip section 100 from a distal direction, along the longitudinal axis A.

The fourth and fifth planes P₄, P₅ are arranged at a set of two symmetrical and combined angles, such that the fourth and fifth planes P₄, P₅ are symmetrically arranged in relation to the longitudinal axis, and also to the first and second planes P₁, P₂. The symmetrical angles of the fourth and fifth planes P₄, P₅ are thus comprised of two combined angles measured in different planes or views. As seen in FIG. 7D, when the tip section 100 is viewed from a top view, or along plane P₂, a first angle phi φ may be measured on either side of the second plane P₂ in the first plane P₁.

The second component of the arrangement of the fourth and fifth planes P_{4 ,}P₅ is a rotational angle omega ω around longitudinal axis A, as illustrated in FIG. 7E. FIG. 7E illustrates a tip section 100 as seen along the longitudinal axis A. The fourth and fifth planes P₄, P₅ are defined by rotation in two opposite directions in relation to the reference planes. Thus, the two angles phi φ and omega ω together describe the arrangement of fourth and fifth planes P₄, P₅, and thus the angles defining the two secondary facets F₂ and F₃.

As is evident from the above and seen in the figures, the two secondary facets F_{2,} F₃ form the distal tip 6 together with an outer mantle surface of the tip section 100. Hence, the sharpness of the distal tip may be controlled by both phi φ and omega ω, thereby offering the ability to optimize sharpness and finding the most effective penetration of e.g. a tissue.

It has been found by the inventors that if phi φ is larger than theta θ, a more suitable geometry is obtained. However, if phi φ is larger than 45 degrees, the tip becomes too blunt.

In addition, especially when providing the one primary facet F₁ at angle theta θ and two secondary facets F₂ and F₃, at angles phi φ and omega ω as described above, it is apparent that theta must be low, preferably under 30 degrees, to obtain a usable tip at all. However, as is described in the experiments below, if theta θ is under 10 degrees, then the tip will have an unsatisfactory rigidity.

Thus, through extensive testing, calculation and inspection of resulting tips the present inventors have reached the conclusion that in order to obtain an improved tip for penetration of a blood vessel wall and surrounding tissue with minimal trauma, leading to minimal bleeding on removal, as well as a needle tip suitable for multiple penetration procedures, the following criteria are preferable. A needle tip section with one primary facet F₁ and two secondary facets F₂ and F₃ is preferably provided at primary facet angle theta θ being between 10.0 and 20.0 degrees, and secondary facets provided at +/−phi φ angles between 15.0 and 20.0 degrees, and +/−omega ω angles between 25.0 and 90.0 degrees.

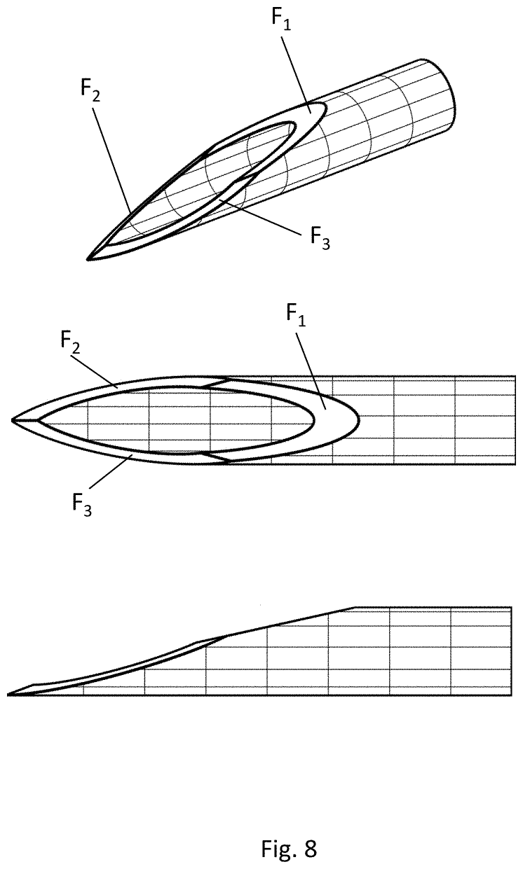

As one example, a needle tip section is shown in FIG. 8, having theta θ angle 12.5 degrees, phi φ angle +/−18.0 degrees, omega angles +/−30.0 degrees.

Another example of a suitable tip section would be a tip having theta θ angle 15.0 degrees, phi φ angle +/−20.0 degrees, omega angles +/−30.0 degrees.

It has thus been found by the inventors, that the combined effect of controlling the intersection between F₂ and F₃ by both phi φ and omega ω as described herein results in a triangular point with optimum sharpness and effective penetration of e.g. tissue, with minimal bleeding.

The above described dimensions and configuration of a tip section 100 works in combination with the overall tapered tip portion 5 of the cannula 1, such that a less-traumatic penetration of a vessel wall may be achieved, mitigating the need to provide any specific closure measures, such as plugging or stopping the hole made by the penetrating tip in the vessel wall.

According to a first aspect, an endoluminal delivery cannula, for delivery of a substance to an extravascular or intraorgan (e.g., intramyocardial) target site via the vascular system of a human or animal is disclosed. The endoluminal delivery cannula comprises a cannula hub provided at a proximal end of the cannula and an elongated proximal portion having an outer diameter, wherein the outer diameter is being constant along essentially the entire length of the proximal portion as measured when the cannula is essentially straight. Further, the cannula comprises a tip portion arranged distally of the proximal portion and extending from the proximal portion to a distal tip of the cannula, and a continuous lumen extending from the proximal end of the cannula through the proximal portion and the tip portion to the distal tip. The tip portion has an opening at the distal tip to provide communication between the lumen and the exterior of the cannula. The tip portion is preferably tapered towards the distal tip by being provided at the proximal end of the tip portion with an outer diameter being essentially the same as the outer diameter of the elongated proximal portion, and an outer diameter at the distal tip being smaller than the outer diameter at the proximal end of the tip portion.

In some aspects, the endoluminal delivery cannula has a distal tip with a pointed tip section for penetrating tissue, wherein the pointed tip section comprises at least one primary facet and two secondary facets, wherein the two secondary facets are arranged proximally of said primary facet.

According to a further aspect, an endoluminal delivery assembly, for delivery of a substance to an extravascular or intraorgan (e.g., intramyocardial) target site via the vascular system of a human or animal body is disclosed. The assembly comprises an endoluminal delivery cannula, a protective catheter adapted for insertion into the vascular system of a human or animal body, wherein a distal end of the assembly is configured to be guided to a position in the vascular system suitable for accessing the intended extravascular or intraorgan (e.g., intramyocardial) target site. The assembly further comprises a proximal catheter hub provided at the proximal end of the protective catheter and adapted for guiding the catheter through the vascular system, wherein the proximal catheter hub is adapted for the endoluminal delivery cannula to be inserted therethrough and into said protective catheter.

According to yet another aspect, a method for delivery of a substance to an extravascular or intramyocardial target site via the vascular system of a human or animal body is disclosed. The method comprises the steps of

a) providing an assembly as disclosed herein,

b) navigating a distal end of the protective catheter to a location near the extravascular or intramyocardial target site,

c) directing the distal end of the protective catheter towards a vessel wall in a general direction of the extravascular or intramyocardial target site,

d) advancing the distal tip of the endoluminal delivery cannula such that a tip of the endoluminal delivery cannula penetrates the myocardium or vessel wall and reaches the extravascular or intramyocardial target,

e) injecting the substance into the extravascular or intramyocardial target site,

f) retracting the distal tip of the endoluminal delivery cannula into the protective catheter.

The endoluminal delivery device can be used to deliver more than one dose of a therapeutic to an intraorgan or extravascular site without harmful effects to the delivery site. Accordingly, in embodiments, for repeating dosing with a therapeutic, the above method is repeated with multiple doses. In one embodiment, the device is reloaded with a dose of therapeutic before each injection. In another embodiment, the device is loaded with multiple doses of the therapeutic, which is then injected in separate doses at the intraorgan or extravascular delivery site.

In another aspect, the disclosure provides an endoluminal delivery cannula (1), for delivery of a substance to an extravascular or intramyocardial target site via the vascular system of a human or animal body,

said cannula (1) having a proximal end (2) configured to remain outside the body and a distal end (3) configured to be inserted into the body via the vascular system to access the extravascular or intramyocardial target site, said cannula (1) having a total longitudinal length (L₁) from said proximal end (2) to said distal end (3), and said cannula (1) comprising

a cannula hub (8) provided at the proximal end (2) of the cannula (1),

an elongated proximal portion (4) having a longitudinal length (L₂) and an outer diameter (D₁), said outer diameter (D₁) being constant along essentially the entire length of the proximal portion (4) as measured when the cannula is essentially straight, and

a tip portion (5) arranged distally of the proximal portion (4) and extending from the proximal portion (4) to a distal tip (6) of the cannula (1), and

a continuous lumen (7) extending from the proximal end (2) of the cannula through the proximal portion (4) and the tip portion (5) to the distal tip (6) and said lumen (7) having an inner diameter (D₂) along the entire length of the cannula (1),

said tip portion (5) having a primary opening (9) at the distal tip (6) to provide communication between the lumen (7) and the exterior of the cannula (1),

said tip portion (5) optionally being tapered towards the distal tip (6) by being provided at the proximal end of the tip portion (5) with an outer diameter (D₃) being essentially the same as the outer diameter (D₁) of the elongated proximal portion (4), and an outer diameter (D₄) at the distal tip (6) being smaller than the outer diameter (D₃) at the proximal end of the tip portion (5).

In embodiments, the total longitudinal length (L₁) from said proximal end (2) to said distal end (3) is within in the range of approximately 300 mm to 2500 mm.

In embodiments, the tip portion (5) is tapered along the entire tip portion towards the distal tip (6) by being provided at the proximal end of the tip portion (5) with an outer diameter (D₃) being essentially the same as the outer diameter (D₁) of the elongated proximal portion (4), and an outer diameter (D₄) at the distal tip (6) being smaller than the outer diameter (D₃) at the proximal end of the tip portion (5).

In embodiments, the tapered tip portion (5) has a longitudinal length (L₃) within the range of 5 mm to 300 mm. In embodiments, the tapered tip portion (5) has a longitudinal length (L₃) of at least 100 mm, preferably between 100 mm and 300 mm, more preferably between 200 mm and 280 mm.

In embodiments, the outer diameter D₄ at the distal tip (6) is within the range of 0.10 mm to 0.25 mm, and preferably between 0.15 mm to 0.22 mm.

In embodiments, the tip portion (5) and/or proximal portion (4) is provided with one or several radiopaque marker bands (11) at predefined distances from the distal tip (6).

In embodiments, the tip portion (5) is provided with one or several protruding depth limit elements (12).

In embodiments, the tip portion (5) is further provided with one or several side openings (9′) along at least part of the tip portion (5).

In embodiments, the the cannula hub (8) is provided with an internal longitudinal channel (10) and a female connector (13) at its proximal end, said internal longitudinal channel (10) configured to provide communication between the continuous lumen (7) of the cannula and the female connector (10), wherein the internal longitudinal channel (10) and the female connector (13) together have a total inner volume being less than 0.45 ml when a corresponding male connector is attached to the female connector (13).

In embodiments, the tip portion (5) is provided with a distal pointed tip section (100) for penetrating tissue, said pointed tip section (100) comprising at least one primary facet (F₁) and two secondary facets (F₂, F₃), wherein said two secondary facets (F₂, F₃) are arranged proximally of said primary facet (F₁).

In embodiments, the distal tip section (100) having

a first plane (P₁) being arranged along a central longitudinal axis (A), and a second plane (P₂) being arranged along the longitudinal axis (A), said first and second planes (P₁, P₂) being perpendicular to each other, and

a third plane (P₃) being positioned at an angle theta (θ), in respect to the first plane (P₁), said third plane (P₃) being symmetrically arranged in relation to the second plane (P₂), and

a fourth plane (P₄) and fifth plane (P₅) being arranged at a set of two symmetrical angles, said angles being symmetrical in relation to each of said first and second planes (P₁, P₂),

said symmetrical angles being combined angles comprising a first angle phi (φ) as measured from the second plane (P₂) in the first plane (P₁), and a second angle omega (ω) being a rotational angle around longitudinal axis (A),

wherein

said primary facet (F₁) of said distal tip section (100) is provided in said third plane (P₃), and

said two secondary facets (F₂, F₃) are provided in said fourth and fifth plane (P_{4 ,}P₅), respectively.

In embodiments, said two secondary facets (F₂, F₃) form a distal tip (6) together with an outer mantle surface of the tip section (100).

In embodiments, the angle phi (φ) is larger than said angle theta (θ).

In embodiments, said one primary facet (F₁) and two secondary facets (F₂, F₃) are provided at primary facet angle theta (θ) being between 10.0 and 20.0 degrees, and secondary facets provided at +/−phi (φ) angles between 15.0 and 20.0 degrees, and +/−omega (ω) angles between 25.0 and 90.0 degrees.

In another aspect, the disclosure provides an endoluminal delivery assembly (400), for delivery of a substance to an extravascular or intramyocardial target site via the vascular system of a human or animal body, comprising

the endoluminal delivery cannula (1) according to any of claims 1 to 15,

a protective catheter (150) adapted for insertion into the vascular system of a human or animal body,

wherein a distal end of said assembly is configured to be guided to a position in the vascular system suitable for accessing the intended extravascular or intramyocardial target site, and

a proximal catheter hub (160) provided at the proximal end of the protective catheter (150) and adapted for guiding the protective catheter (150) through the vascular system,

said proximal catheter hub (160) being adapted for the endoluminal delivery cannula (1) to be inserted therethrough and into said protective catheter (150).

In embodiments, the catheter hub (160) further comprises a locking means (162) adapted to reversibly alternate between a locked state and an unlocked state, said locking means (162) configured, in a locked state, to prevent axial movement between the protective catheter (150) and the endoluminal delivery cannula (1).

In embodiments, the locking means (162) comprises a locking wheel (164) provided with internal threads (165a) collaborating with external threads (165b) of the catheter hub housing, said the locking means adapted such that when said locking means is actuated, an internal locking gasket (166) is compressed to grip a proximal end of said endoluminal delivery cannula.

In embodiments, the disclosure provides a method for delivery of a substance to an extravascular or intraorgan (e.g., intramyocardial) target site via the vascular system of a human or animal body, the method comprising the steps of

a) providing an endoluminal delivery cannula assembly according to the disclosure;

b) navigating a distal end of said protective catheter to a location near said extravascular or intraorgan (e.g., intramyocardial) target site,

c) directing said distal end of said protective catheter towards a vessel wall in a general direction of said extravascular or intraorgan (e.g., intramyocardial) target site,

d) advancing the distal tip of said endoluminal delivery cannula such that a tip of said endoluminal delivery cannula penetrates the intraorgan (e.g., myocardium) or vessel wall and reaches said extravascular or intraorgan (e.g., intramyocardial) target site,

e) injecting said substance into said extravascular or intraorgan (e.g., intramyocardial) target site,

f) retracting said distal tip of said endoluminal delivery cannula into said protective catheter.

In embodiments, the method further comprises the step of reversibly locking a locking means during at least steps b) and c), such that axial displacement by the guide catheter and the endoluminal delivery cannula are reversibly prevented in relation to each other.

In embodiments, the locking means comprises a locking wheel provided with internal threads collaborating with external threads of a catheter housing, wherein when the locking means is actuated, a gasket is compressed to grip a proximal end of said endoluminal delivery cannula.

In embodiments, the endoluminal delivery cannula comprises one or several outwardly protruding depth limit elements, and said depth limit elements provide a resistance that is felt by the user when a depth limit elements reaches the vessel or myocardial wall.

In embodiments, the method further comprises the step of repeating all or some of steps b) to f).

### II. Therapeutics

The methods and compositions of the disclosure can be used to deliver a wide variety of therapeutics to an intraorgan or extravascular site of a subject. As used herein, the terms “therapeutic” and “therapeutic agent” are used interchangeably to refer to a composition that has a beneficial effect in a subject and are intended to encompass both cell-based therapeutics and non-cell-based therapeutics, such as protein-based therapeutics and oligonucleotide-based therapeutics.

As used herein, a “cell-based therapeutic” refers to an agent intended for therapy that comprises viable (i.e., living) cells and is intended to encompass isolated cells, cell suspensions, cell cultures, naturally-occurring cells and non-naturally-occurring cells including modified cells (e.g., recombinantly-modified cells). In an embodiment, the cells are stem cells, non-limiting examples of which include embryonic stem cells, induced pluripotent stem cells and adult stem cells. In an embodiment, the cells are stem cell-derived progenitor cells. In an embodiment, the cells are mesenchymal stem cells (MSCs). In an embodiment, the cells are cardiac progenitor cells. In an embodiment, the cells are human cardiac ventricular progenitor (HVP) cells. In an embodiment, the cells are kidney progenitor cells. In an embodiment, the cells are pancreatic progenitor cells (e.g., islet beta progenitor cells). In an embodiment, the cells are neural progenitor cells. In an embodiment, the cells are ocular progenitor cells. In an embodiment, the cells are liver progenitor cells. In an embodiment, the cells are kidney progenitor cells.

In an embodiment, the cells are mesenchymal stem cells. MSCs can be obtained from fetal or adult tissue sources, including but not limited to amniotic fluid/membranes, placental or fetal membranes, umbilical cord, bone marrow, adipose tissue and dental tissue. Moreover, human mesenchymal stem cells are commercially available, such as Poetic S™ Normal Human Bone Marrow Derived Mesenchymal Stem Cells (Lonza, Catalog #PT-2501).

In an embodiment, the stem cell-derived progenitor cells comprise cardiac progenitor cells. The use of stem cells, including ES cells, iPSCs and adult stem cells, to generate progenitor cells that are committed to the cardiac lineage (i.e., progenitor cells that differentiate into cardiac cells) has been described extensively in the art (see e.g., U.S. Patent Publication 2004018004; U.S. Patent Publication 20050214260; U.S. Patent Publication 20060246446; U.S. Patent Publication 20100166714; U.S. Patent Publication 20110033430; U.S. Patent Publication 20130189785; Jackson, K. A. et al. (2001) J. Clin. Invest. 107:1395-1402; Cai, C. L. et al. (2003) Dev. Cell. 5:877-889; Moretti, A. et al. (2006) Cell 127:1151-1165; Qyang, Y. et al. (2007) Cell Stem Cell. 1:165-179; Kwon, C. et al. (2007) Proc. Natl. Acad. Sci. USA 104:10894-10899; Bu, L. et al. (2009) Nature 460:113-117; Lian, X. et al. (2012) Proc. Natl. Acad. Sci. USA 109:E1848-57; and Lian, X. et al. (2013) Nat. Protoc. 8:162-175).

In a preferred embodiment, the cardiac progenitor cells are human ventricular progenitor cells (HVPs), which are biased toward differentiating into cardiac ventricular myocytes. Methods for generating HVPs have been described in the art (e.g., Examples 1 and 10 of U.S. Patent Publication 2019/0062696. Suitable hPSC starting cells include iPSC and human embryonic stem cells, such as ES cell lines. Briefly, for the protocol, Wnt/β-catenin signaling first is activated in the hPSCs, followed by an incubation period, followed by inhibition of Wnt/β-catenin signaling. Wnt/β-catenin signaling activation is achieved by incubation with a Gsk3 inhibitor, such as CHIR98014 (CAS 556813-39-9; commercially available from, e.g., Selleckchem). Wnt/β-catenin signaling inhibition is achieved by incubation with a Porcn inhibitor, such as Wnt-C59 (CAS 1243243-89-1; commercially available from, e.g., Selleckchem or Tocris). The Gsk3 inhibitor is used to promote cardiac mesodermal differentiation, whereas the Porcn inhibitor is used to enhance ventricular progenitor differentiation from mesoderm cells.

To generate a culture comprising HVPs, hPSCs are cultured on day 0 in a medium comprising a Gsk3 inhibitor, such as CHIR-98014, for at least 24 hours, followed by culturing the hPSCs in a medium comprising a Porcn inhibitor, such as Wnt-C59 (and lacking the Gsk3 inhibitor) starting on day 3 of culture and continuing for at least 48 hours (e.g., days 3-5) such that HVPs are generated. Experiments showed that after 24-hours of treatment with CHIR-98014, more than 99% of hPSCs expressed the mesoderm marker Brachyury, and three days later after treatment with CHIR-98014, more than 95% of differentiated cells expressed Mesp1, which marks the cardiac mesoderm. Furthermore, 48-hour treatment with Wnt-C59 enhanced ventricular progenitor differentiation from mesoderm cells. HVP generation is optimal between days 5 and 8 (inclusive) in culture and typically peaks at day 6 of culture.

Accordingly, as used herein a culture of “day 5-8 cardiac progenitor cells” or “day 6 cardiac progenitor cells” refers to a culture in which hPSCs have been subjected to activation of Wnt/β-catenin signaling (e.g., by culture with a Gsk3 inhibitor) starting on day 0 of culture for at least 24 hours, followed by inhibition of Wnt/β-catenin signaling (e.g., by culture with a Porcn inhibitor) from day 3 to day 5 of culture, such that the culture contains HVPs on days 5-8. The generation, characterization and use of HVPs, including surface marker identification and methods of transplanting are described in detail in, for example, U.S. Patent Publication 20160053229; U.S. Patent Publication 20160108363; U.S. Patent Publication 20170240964; U.S. Patent Publication 20180148691; U.S. Patent Publication 20190062696.

In another embodiment, the cells comprises pancreatic cells or progenitors thereof. In one embodiment, the pancreatic cells are islet beta cells, or progenitors thereof. In one embodiment, the pancreatic cells are islet alpha cells, or progenitors thereof. The use of stem cells, including ES cells, to generate progenitor cells that are committed to the pancreatic cell lineage has been described extensively in the art.

For example, generation and use (including transplantation) of islet beta cells, and progenitors thereof, is described in, for example, Pagliuca and Melton (2013) Develop. 140:2472-2483; Ma et al. (2018) Proc. Natl. Acad. Sci. USA 115:3924-3929; Zhou and Melton (2018) Nature 557:351-358; US Patent Publication 20130344594; US Patent Publication 20150231181; US Patent Publication 20160326494; US Patent Publication 20160175363; US Patent Publication 20161777267; US Patent Publication 20161777268; US Patent Publication 20161777269; US Patent Publication 20170029778; US Patent Publication 20200199539; and US Patent Publication 202000347358.

More recently, generation of stem cell-derived human pancreatic alpha cells from pluripotent stem cells via a transient pre-alpha cell intermediate has been described (Peterson et al. (May 7, 2020) Nature Comm. 11:Article Number 2241). With respect to treatment of Type I diabetes using stem cell-derived pancreatic cells, while focus primarily has been on generating insulin-secreting beta cells, there is also evidence indicating that glucagon secreting alpha cells are also involved in the disease progression and proper glucose control. Accordingly, in one embodiment, the pancreatic cells are islet alpha cells, or progenitors thereof.

In another embodiment, the cells comprises neural cells or progenitors thereof. In one embodiment, the neural cells are dopaminergic neural cells, or progenitors thereof. In one embodiment, the neural cells are cholinergic neural cells, or progenitors thereof. The use of stem cells to generate progenitor cells that are committed to the neural lineage (e.g., neural progenitor cells that differentiate into dopaminergic neurons or cholinergic neurons) has been described extensively in the art. For example, stem cell-derived neural progenitors are described and/or reviewed in, e.g., Ochalek et al. (2016) Stem Cell Int. 2016:5838934; Dhivya et al. (2017) Stem Cell Investig. 4:59; DiSanto et al. (2018) Brain Circ. 4:139-141; as well as in US Patent Publication 20090258421; US Patent Publication 20100323444; US Patent Publication 20120122109; US Patent Publication 20130089926; US Patent Publication 20170266235; US Patent Publication 20170296582; US Patent Publication 20190024047; and US Patent Publication 20200024572.

In another embodiment, the cells comprises ocular cells or progenitors thereof. In one embodiment, the ocular cells or progenitors are limbal stem cells (also known as corneal epithelial stem cells). In one embodiment, the ocular cells are retinal pigment epithelial cells (RPE) or progenitors thereof. The use of stem cells, including ES cells, iPSCs and adult stem cells, to generate progenitor cells that are committed to an ocular lineage (i.e., progenitor cells that differentiate into ocular cells such as corneal epithelial cells or retinal pigment epithelial cells) has been described extensively in the art.

For example, stem cell-derived ocular progenitors are described and/or reviewed in, e.g., Casaroli-Marano et al. (2015) J. Clin. Med. 4:318-342; Atallah et al. (2016) Clin. Ophthamol. 10:593-602; Gokuladhas et al. (2017) Genes Dis. 4:88-99; as well as in US Patent Publication 20070196919; US Patent Publication 20110223140; US Patent Publication 20140186309; US Patent Publication 20160251618; US Patent Publication 20170321188; US Patent Publication 20180327713; US Patent Publication 20180333459; and US Patent Publication 20200362301.

In embodiments, the cells are modified to express one or more agents, e.g., one or more therapeutic agents. Modified cells include cells recombinantly modified with DNA and cells recombinantly modified with RNA (e.g., mRNA). In one embodiment, the modified cells are modified MSCs that harbor one or more mRNA constructs such that they express one or more agents of interest encoded by the mRNA. Modified MSCs expressing mRNA constructs have been described in the art, for example in US Patent Publication 2021/0052776, the entire contents of which is hereby specifically incorporated by reference.

In embodiments, the therapeutic is a protein-based agent. Non-limiting examples of protein-based agents include cytokines, chemokines, growth factors, antibodies (including human, humanized, chimeric, single chain and bispecific antibodies) and nanobodies. Protein-based therapeutics also include proteinaceous agents that are incorporated into a delivery system, such as a microparticle or lipid nanoparticle.

In embodiments, the therapeutic is an oligonucleotide-based agent. In one embodiment, the oligonucleotide-based agent is a DNA agent, non-limiting examples of which include plasmids, DNA expression vectors and DNA-based viral vectors. In one embodiment, the oligonucleotide-based agent is an RNA agent, non-limiting examples of which include mRNA agents and antisense oligonucleotide compounds. In one embodiment, the RNA agent is a modified mRNA agent. Other non-limiting examples of oligonucleotide-based agents include small interfering RNAs (siRNA), microRNAs (miRNA), ribozymes, immune stimulating nucleic acids, antagomirs, antimirs, microRNA mimics, supermirs, UI adaptors, aptamers, and CRISPR gene-editing machinery. Oligonucleotide-based therapeutics also include DNA-based or RNA-based agents that are incorporated into a delivery system, such as a microparticle or lipid nanoparticle.

### III. Delivery of Therapeutics

The methods and compositions of the disclosure are useful for delivery of therapeutics to an intraorgan or extravascular site of a subject. The methods involve providing an endoluminal delivery cannula comprising a dose of the therapeutic and administering the dose of the therapeutic to the intraorgan or extravascular site in the subject using the endoluminal delivery cannula.

As used herein, an “intraorgan” site refers to a location within the tissue of an organ, such as an intramyocardial site within the heart or an intracapsular site within the kidney. As used herein, an “extravascular” site refers to a site outside the vascular system but that is accessed through the vascular system using the endoluminal delivery device as described herein.

In embodiments, the methods are used to deliver a therapeutic to a site within the heart, such as an intramyocardial site. In an embodiment, the therapeutic is delivered to a ventricular site. In an embodiment, the therapeutic is delivered to an atrial site. In an embodiment, the therapeutic is delivered to the cardiac apex. In an embodiment, the therapeutic is delivered to a septal site.

In embodiments, the methods are used to deliver a therapeutic to a site within the kidney, such as a subcapsular site.

In embodiments, the methods are used to deliver a therapeutic to a site within the pancreas.

In embodiments of the delivery method, the dose is retained at the intraorgan or extravascular site at a level higher than an equivalent dose administered using a 26- or 27-gauge needle. While not intending to be limited by mechanism, both the inner diameter (ID) of the lumen of the endoluminal delivery device (or “Extroducer” as referred to herein) and the outer diameter (OD) of the device, are significantly smaller than that of a 26- or 27-gauge needle, as illustrated in the table below, which may contribute to the effectiveness of the retention of therapeutics delivered using the endoluminal delivery device.

| | Inner Diameter (ID) | Outer Diameter (OD) |
|---|---|---|
| Device | (in micrometers) | (in micrometers) |
| 26-gauge needle | 260 | 464 |
| 27-gauge needle | 210 | 413 |
| Extroducer | 140-160* | 250** |

*ID = inner diameter of lumen of Extroducer
**OD = outer diameter of elongated proximal portion of Extroducer, as well as outer diameter D₃ of the proximal end of the tip portion

In various embodiments, the OD of the elongated proximal portion of the endoluminal delivery device, as well as the outer diameter D₃ of the proximal end of the tip portion, is smaller than the OD of a 26- or 27-gauge needle. In various embodiments, the ID (lumen) of the endoluminal delivery device is smaller than the ID of a 26- or 27-gauge needle.

In various embodiments, the dose is retained at a level at least 2-fold, or at least 3-fold, or at least 4-fold, or at least 5-fold, or at least 6-fold, or at least 7-fold, or at least 8-fold or at least 9-fold higher than an equivalent dose administered using a 26- or 27-gauge needle. The level of retention of a dose can be determined by, for example, labeling the therapeutic, administering the therapeutic via the endoluminal delivery device and via a 26- or 27-gauge needle and comparing retention of the labeled therapeutics, e.g., as described in Example 3.

In embodiments, the dose of the therapeutic is administered within a particular time period (e.g., the therapeutic is passed through the endoluminal delivery device at a specified speed or passage rate). For examples, doses of cells can be effectively passed through the Extroducer with high cellular recovery and viability within a time period of 4 minutes or less, more preferably 3 minutes or less, even more preferably 2 minutes or less. Moreover, as discussed in Example 2, passing cells through the Extroducer at speeds even faster than 2 minutes (e.g., 30, 60 or 90 seconds) generally increased the viability of the cells as compared to slower speeds (e.g., 2-4 minutes). While not intending to be limited by mechanism, it is believed the shearing stress, as well as clumping and catheter adherence, on the cells is higher with slower speeds. Accordingly, in embodiments, the dose of the therapeutic (e.g., dose of cells) is administered in 2 minutes or less, 90 seconds or less, 60 seconds or less, 30-60 seconds, 30-90 seconds, 60-90 seconds, within 90 seconds, within 60 seconds or within 30 seconds.

A “dose” of a therapeutic agent is intended to refer to the amount of agent loaded into the endoluminal delivery device for delivery to the intraorgan or extravascular site. A dose typically is contained within a suitable volume for loading into the endoluminal delivery device. In embodiments, the volume of the dose can be, for example, 50-500 μL, 100-300 μL, 150-250 μL or 200 μL.

In embodiments wherein the therapeutic comprises cells, the dose can include a concentration of cells in a range of 10,000-1,000,000 cells/μl, 10,000-500,000 cells/μl, 10,000-250,000 cells/μl, 25,000-500,000 cells/μl, 25,000-250,000 cells/μl, 33,000-200,000 cells/μ1, 50,000-500,000 cells/μl, 50,000-200,000 cells/μl, 100,000-300,000 cells/μl, 150,000-250,000 cells/μl, 10,000 cells/μl, 25,000 cells/μl, 33,000 cells/μl, 50,000 cells/μl, 66,000 cells/μ1, 100,000 cells/μl, 133,000 cells/μl, 150,000 cells/μl, 200,000 cells/μl, 250,000 cells/μl, 300,000 cells/μl, 350,000 cells/μl, 400,000 cells/μl, 450,000 cells/μl, or 500,000 cells/μ1.

In embodiments, a dose of cells comprises at least 10 million cells, at least 20 million cells, at least 30 million cells or at least 40 million cells, for example in a volume of 50-500 μL, 100-300 μL, 150-250 μL or 200 μL. In one embodiment, a dose of cells comprises 100,000-200,000 cells/μL in a volume of 200 μL for a total cell amount of 20-40 million cells.

In various embodiments, the flow rate of the dose through the endoluminal delivery device is 20-2000 μl/minute, 20-1000 μl/minute, 20-500 μl/minute, 50-1000 μl/minute, 50-500 μl/minute, 100-1000 μl/minute, 100-500 μl/minute, 100-300 μl/minute, 50-200 μl/minute, 20 μl/minute, 50 μl/minute, 100 μl/minute, 150 μl/minute or 200 μl/minute. In an embodiment, the therapeutic comprises cells at a concentration of 100,000-200,000 cells/μl (e.g., 133,000 cells/μl) that are passaged through the endoluminal delivery device at a flow rate of 50-200 μl/minute (e.g., the cell dose is in a volume of 200 μl and it is passaged through the device within one minute).

In embodiments, conditions (e.g., speeds of flow) are selected that allow for passage of cells through the Extroducer with a resulting cell yield out of the Extroducer of at least 40%, at least 45%, at least 50%, at least 55%, at least 60%, at least 65%, at least 70%, or at least 75% of the starting cell amounts. In embodiments, conditions (e.g., speeds of flow) are selected that allow for passage of cells through the Extroducer with a resulting cell viability out of the Extroducer of at least 40%, at least 45%, at least 50%, at least 55%, at least 60%, at least 65%, at least 70%, or at least 75% of the starting cell amounts. Measurement of cell yield and cell viability after passage through the Extroducer is described further in Examples 1 and 2.

In an embodiment, for an optimal balance of cell viability and cell yield (the proportion of cells surviving coming out of the Extroducer), a cell concentration of 133,000 cells/μL is used in a volume of 200 μ,L (for a cell dose of 26.6 million cells) at a passage speed of 60 seconds, which resulted in approximately 70% cell yield and 70% cell viability.

### EXAMPLES

Below are examples of specific embodiments for carrying out the present invention. The examples are offered for illustrative purposes only and are not intended to limit the scope of the present invention.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

Unless otherwise stated, all reagents and chemicals were obtained from commercial sources and used without further purification.

### Example 1

### Passage of Mesenchymal Stem Cells through Extroducer In Vitro

In this example, the effect of passaging human mesenchymal stem cells (MSCs) through the Extroducer in vitro was examined. The MSCs were first modified by electroporation with modified mRNA encoding green fluorescent protein (GFP)(5 μg) and modified mRNA encoding vascular endothelial growth factor (VEGF)(5 μg) and cryopreserved. Following thawing, the modified MSCs were either immediately seeded on culture vessels or were passed through the Extroducer before being seeded.

In a first set of experiments, 1×10⁶ cells in 100 μl media were passaged through the Extroducer. Test #1, in which the cells were passed through the Extroducer for 3 minutes and 47 seconds, resulted in 72% cell recovery and 72% cell viability. Test #2, in which cells were passed through the Extroducer for 3 minutes and 10 seconds, resulted in 77% cell recovery and 75% cell viability. Since the thawed MSCs that were immediately plated exhibited 75% viability, the results from this first set of experiments indicated that passage of the MSCs through the Extroducer did not significantly affect cell viability.

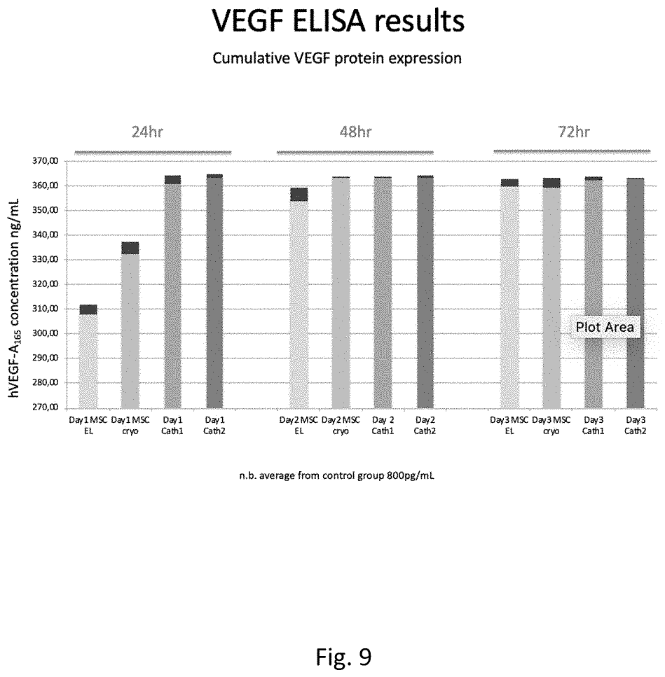

Production of VEGF by the MSCs was examined by standard ELISA. Representative results for cumulative VEGF production at 24 hours, 48 hours and 72 hours are shown in FIG. 9. The results showed that the level of VEGF production by the MSCs passed through the Extroducer was actually higher at 24 hours than the level of VEGF production by MSCs assayed after electroporation or post-thaw after cryopreservation, with VEGF production by all four samples being essentially equivalent by 72 hours. Thus, these results indicated that passage of the MSCs through the Extroducer did not affect the ability of the MSCs to produce a heterologous protein encoded by an introduced nucleic acid.

In a second set of experiments, MSCs were again passaged through the Extroducer but using increased volumes and cell doses. Test #3 used 4×10⁶ cells in 200 μl media, with the first 100 μl passaged in 2 minutes, 30 seconds, and the second 100 μl passaged in 2 minutes, 35 seconds. The recovery yield was 80%, versus 95% viability for the freshly thawed MSCs. Test #4 used 1×10⁶ cells that were left to incubate in 200 μl carrier for 3.5 hours at room temperature. Carriers tested were saline and PBS. The saline carrier resulted in a cell yield of 77% and a cell viability of 80%. The PBS carrier resulted in a cell yield of 72% and a cell viability of 83%. Thus, the results indicated that use of increased cell doses, increased volumes, different types of carriers and/or incubating the cells at room temperature for several hours before passaging did not significantly affect cell viability.

In summary, this example demonstrates that modified MSCs can be passaged through the Extroducer in vitro without negatively impacting either cell viability or the ability of the cells to secrete an mRNA-encoded protein.

### Example 2

### Passage of Human Ventricular Progenitor Cells through Extroducer In Vitro

In this example, the effect of passaging human ventricular progenitor cells (HVPs) through the Extroducer in vitro was examined. HVP cells were obtained by differentiation of human embryonic stem (hES) cells according to published protocols (e.g., Examples 1 and 10 of US Patent Publication 2019/0062696). HVPs were harvested at day 8 of the differentiation protocol, at which stage the cells were approximately 80% ISL1+.

Frozen cells were thawed and passed through the Extroducer catheter in 200 μl volumes in a media carrier (RPMI +Insulin). The HVPs were tested at four different concentrations, as summarized below in Table 1:

TABLE 1

| Cell Concentration (×1000/μl) | Volume (μl) | Cells (Millions) |
|---|---|---|
| 200 | 200 | 40 |
| 133 | 200 | 26.6 |
| 66 | 200 | 13.2 |
| 33 | 200 | 6.6 |

Each cell concentration was passaged through the Extroducer at two different speeds, 2 minutes vs. 4 minutes. Each cell concentration was passaged through the catheter a minimum of 2 times at each speed, with cell viability and cell yield being measured and averaged after passage. The results are summarized below in Table 2, with viability, yield and standard deviations (SD) shown as percentages:

TABLE 2

| Cell | | | | | | | | |
|---|---|---|---|---|---|---|---|---|
| Concentration | 2 minutes | | | | 4 minutes | | | |
| (×1000/ml) | Viability | SD | Yield | SD | Viability | SD | Yield | SD |
| 200 | 63.7 | 2.8 | 72.2 | 2.9 | 55.6 | 8.8 | 39.8 | 23.6 |
| 133 | 57.4 | 2.1 | 71.6 | 2.3 | 56.8 | 3.9 | 62.7 | 3.6 |
| 66 | 61.0 | 5.3 | 71.1 | 11.9 | 59.3 | 4.2 | 56.7 | 7.3 |
| 33 | 47.8 | 4.8 | 77.7 | 2.3 | 43.4 | 4.7 | 61.1 | 6.1 |

Additionally, the higher doses (40 million cell group and 26.6 million cell group) were also passaged at quicker speeds of 30 seconds, 60 seconds and 90 seconds. These higher doses were chosen for further characterization as these higher cell amounts are likely to be needed in clinical settings. The results are summarized below in Table 3, with viability (via.), yield and standard deviations (SD) shown as percentages:

TABLE 3

| Cell Conc. | 30 seconds | | | | 60 seconds | | | | 90 seconds | | | |
|---|---|---|---|---|---|---|---|---|---|---|---|---|
| (×1000/ml) | Via. | SD | Yield | SD | Via. | SD | Yield | SD | Via. | SD | Yield | SD |
| 200 | 70.78 | 5.42 | 62.89 | 4.77 | 66.67 | 0.88 | 65.94 | 5.50 | 65 | 1 | 78 | 4 |
| 133 | 82.44 | 3.10 | 75.16 | 1.41 | 80.11 | 2.46 | 86.46 | 2.05 | 59 | 4 | 83 | 4 |

Overall, the results demonstrated that all cell concentration groups of HVPs survived passage through the Extroducer well at all passage speeds tested. Passing the cells at faster speeds (e.g., 30, 60 or 90 seconds) generally increased the viability of the cells as compared to slower speeds (e.g., 2 minutes or 4 minutes). It is believed the shearing stress on the cells and catheter adherence of the cells is higher with slower speeds. For the best possible balance of cell viability and cell yield (the proportion of cells surviving coming out of the Extroducer), the cell concentration of 133,000 cells/μL performed best at a passage speed of 60 seconds, which resulted in approximately 70% cell yield and 70% cell viability.

### Example 3

### Use of Extroducer to Deliver Mesenchymal Stem Cells to Heart In Vivo

In this example, the engraftment of mesenchymal stem cells (MSCs) into swine heart tissue following in vivo delivery using the Extroducer was examined. Healthy naïve pigs, with no immunosuppression, were used as the recipients. To evaluate the retention of the MSCs in the swine heart, MSCs radiolabeled with Zr89 were delivered into the cardiac apex of healthy pigs using the Extroducer (n=3). For comparison, radiolabeled MSCs were also delivered into the cardiac apex of healthy pigs using a 26 gauge needle (n=3). All animals were followed for five days post-injection. Gamma counter measurements were performed to determine the % retention of the injected dose (ID). The results are summarized below in Table 4, with radioactivity measured in Megabecquerel (MBq):

TABLE 4

| | | Injected Dose | Gamma Counter | |
|---|---|---|---|---|
| Animal | Device | (MBq) | MBq | % ID |
| 1 | Extroducer | 0.7 | 0.3 | 44% |
| 2 | | 1.2 | 0.4 | 38% |
| 3 | | 1.3 | 0.2 | 16% |
| 4 | 26 Gauge | 4.1 | 0.2 | 6% |
| 5 | Needle | 1.4 | 0.0 | 0% |
| 6 | | 1.2 | 0.1 | 5% |

The results demonstrate that the Extroducer was significantly better than the 26 gauge needle at delivering the MSCs to the cardiac apex in swine hearts, with cell retention being at least 3-fold higher, and as much as 8- to 9-fold higher, in animals treated with the Extroducer versus the 26 gauge needle. Moreover, none of the animals treated with the 26 gauge needle achieved even 10% retention of the injected dose of radiolabeled cells, whereas all of the animals treated with the Extroducer exhibited more than 10% retention of the injected dose of radiolabeled cells. Individual Extroducer-treated animals exhibited greater than 15%, greater than 35% and greater than 40% retention of the injected dose of radiolabeled cells, thereby demonstrating that the Extroducer is capable of delivering cells into the heart such that a significant portion of the delivered cells are retained within the heart.

### Example 4

### Use of Extroducer to Deliver MSCs and mRNA to Heart In Vivo

In this example, additional experiments using the Extroducer to deliver MSCs and mRNA to swine heart were conducted. Healthy naïve pigs, with no immunosuppression, were used as the recipients. The Extroducer was used to deliver one of the following treatments: VEGF-encoding mRNA in a citrate saline formulation (referred to herein as “Naked”), unmodified mesenchymal stem cells (MSCs)(referred to herein as “Naïve treatment”) or MSCs electroporation-loaded with VEGF-encoding mRNA (referred to herein as “Enhanced”). Three pigs were used for each treatment group.

For the cell-treated groups, 3×10⁶ human MSCs were delivered to three sites of cardiac tissue in a 200 μL volume for a total of 9×10⁶ cells delivered in each animal. The mRNA-engineered cells contained a payload of 20 μg modified VEGF mRNA per 1×10⁶ cells (60 μg modified RNA per injection site). For the VEGF mRNA injections in a sucrose-citrate buffer, 60 μg modified RNA doses were used per injection site. After final delivery of cells or mRNA, methylene blue solution (50 μL was administered to the injection site to aid in visual detection upon tissue harvest at study termination. Tissues were harvested 24 hours post-injection.

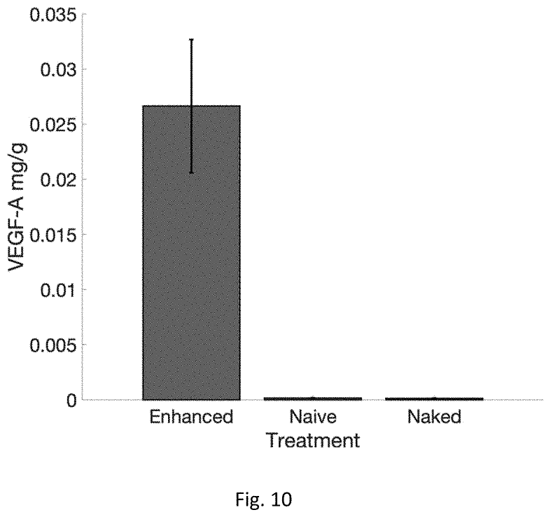

Following harvest, human VEGF protein concentration per gram of cardiac tissue was quantified and compared between the three treatment groups. The results are summarized in FIG. 10. The results demonstrate that treatment with umodified MSCs did not lead to significant VEGF protein production in cardiac tissue, nor did injection of naked VEGF mRNA, whereas injection of MSCs modified with VEGF-encoding mRNA led to expression of large amounts of VEGF protein in the cardiac tissue in vivo. Quantitation determined that the Enhanced treatment led to approximately 200 fold greater VEGF protein expression as compared to the Naked or Naïve treatments.

These experiments demonstrate that the Extroducer was effective in delivering MSCs modified to contain a protein-encoding mRNA to the heart such that the protein encoded by the mRNA was effectively expressed in cardiac tissue in vivo.

### Example 5

### Use of Extroducer to Deliver Mesenchymal Stem Cells to Kidney In Vivo

In this example, the engraftment of mesenchymal stem cells (MSCs) into swine kidney tissue following in vivo delivery using the Extroducer was examined. Healthy naïve pigs, with no immunosuppression, were used as the recipients. Human bone marrow-derived mesenchymal stromal cells (MSCs) were injected using the Extroducer into three sites in the kidney parenchyma in three different animals. Injections were performed so that 3×10⁶ live MSCs were delivered at each injection site. Injections were performed in 30 sec-60s ec in volumes of 200-250 μL. After each MSC injection, the Extroducer was rinsed with 50 μL methylene blue flush in order to stain the injection site.

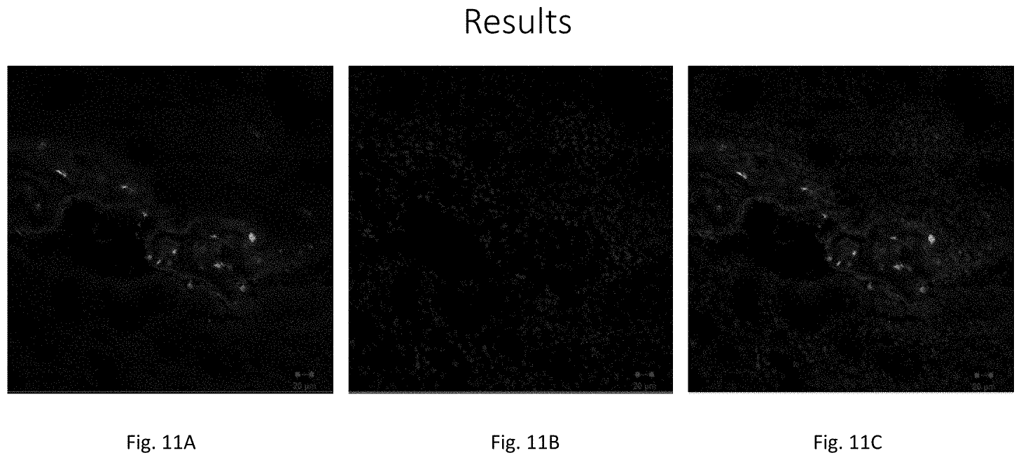

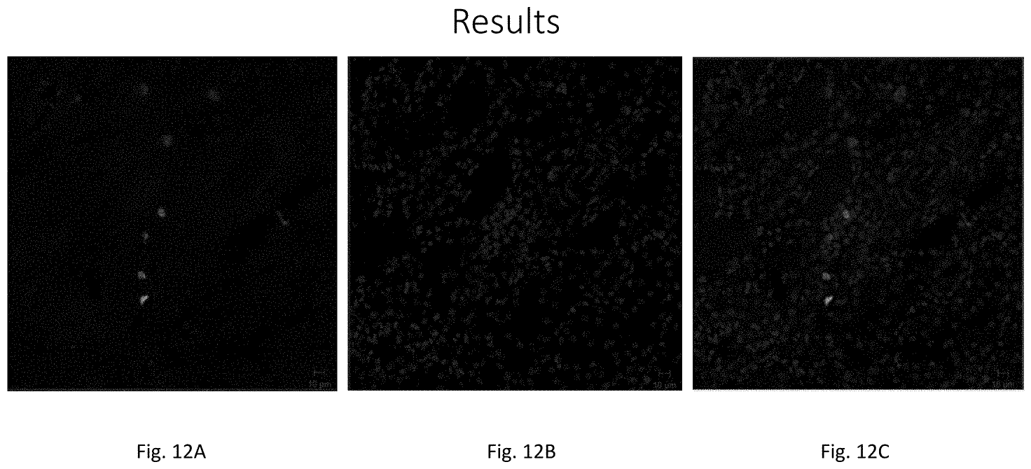

Kidneys were harvested at 24 hours post-injection. Injection sites were found through the visualization of methylene blue detection. Tissue was frozen, cryo-sectioned and stained with human nuclear antigen antibody (anti-NA Ab) to detect and visualize the presence of the human cells in the swine kidney. The results for the kidney cortex are shown in FIGS. 11A-11C. The results for the kidney medulla are shown in FIGS. 12A-12C. The results demonstrate that the Extroducer was able to effectively deliver human MSCs to both the kidney cortex and the kidney medulla in non-immunosuppressed swine.

### INCORPORATION BY REFERENCE

Each patent, publication, and non-patent literature cited in the application is hereby incorporated by reference in its entirety as if each was incorporated by reference individually.

1. A method of delivering a therapeutic to an intraorgan or extravascular site in a subject, the method comprising:
(a) providing an endoluminal delivery cannula comprising a dose of the therapeutic; and
(b) administering the dose of the therapeutic to the intraorgan or extravascular site in the subject using the endoluminal delivery cannula,
wherein the dose is administered in 4 minutes or less; and
wherein the dose is retained at the intraorgan or extravascular site at a level at least 2-fold higher than an equivalent dose administered using a 26- or 27-gauge needle.

2. The method of claim 1, wherein the dose is administered in 3 minutes or less.

3. The method of claim 1, wherein the dose is administered in 2 minutes or less.

4. The method of claim 1, wherein the dose is administered in 90 seconds or less.

5. The method of claim 1, wherein the dose is administered in 60 seconds or less.

6. The method of claim 1, wherein the therapeutic comprises cells at a concentration in the dose of 100,000-300,000 cells/μl.

7. The method of claim 1, wherein the dose is retained at the intraorgan or extravascular site at a level at least 6-fold higher than an equivalent dose administered using a 26- or 27-gauge needle.

8. The method of claim 1, wherein the intraorgan site is within the heart.

9. The method of claim 1, wherein the intraorgan site is within the kidney, the pancreas, the liver, the lungs or the brain.

10. The method of claim 1, wherein the therapeutic comprises stem cells or stem cell-derived progenitor cells.

11. The method of claim 10, wherein the therapeutic comprises mesenchymal stem cells (MSCs).

12. The method of claim 11, wherein the MSCs are modified to express at least one therapeutic agent.

13. The method of claim 12, wherein the MSCs are modified with mRNA encoding the therapeutic agent.

14. The method of claim 10, wherein the therapeutic comprises human ventricular progenitor cells (HVPs) and the intraorgan site is within the heart.

15. The method of claim 1, wherein the therapeutic comprises a protein-based agent.

16. The method of claim 15, wherein the protein-based agent is a nanobody.

17. The method of claim 1, wherein the therapeutic comprises an oligonucleotide-based agent.

18. The method of claim 17, wherein the oligonucleotide-based agent is an mRNA agent.

19. The method of claim 1, wherein the endoluminal delivery cannula comprises:
an elongated proximal portion having an outer diameter (OD),
a tip portion arranged distally of the proximal portion and extending from the proximal portion to a distal tip of the cannula, and
a lumen having an inner diameter (ID) and continuously extending through the proximal portion and the tip portion to the distal tip, the therapeutic being contained within the lumen; wherein
the tip portion has an opening at the distal tip to provide communication between the continuous lumen and the exterior of the cannula; and
the tip portion is tapered towards the distal tip.

20. The method of claim 19, wherein the lumen has an ID of 140-160 micrometers.

21. The method of claim 20, wherein the lumen has an ID of 145-150 micrometers.

22. The method of claim 19, wherein the OD is smaller than the outer diameter of a 26-gauge or 27-gauge needle.

23. The method of claim 19, wherein the OD of the proximal portion is 200-300 micrometers.

24. The method of claim 23, wherein the OD of the proximal portion is 250 micrometers.

25. The method of claim 19, wherein the tip portion has a longitudinal length (L₃) within a range of 5 mm to 300 mm.

26. The method of claim 25, wherein longitudinal length (L₃) of the tip portion is less than 100 mm.

27. The method of claim 25, wherein the tip portion has a proximal end with an outer diameter (D₃) and a distal end at the distal tip with an outer diameter (D₄), wherein the outer diameter (D₃) is the same as the OD of the elongated proximal portion and the outer diameter (D₄) is smaller than the outer diameter (D₃).

28. The method of claim 27, wherein the outer diameter (D₃) is 225-275 micrometers and the outer diameter (D₄) is 175-200 micrometers.

29. The method of claim 27, wherein the outer diameter (D₃) is 250 micrometers and the outer diameter (D₄) is 194 micrometers.

30. The method of claim 19, wherein the dose is administered to the intraorgan or extravascular site in the subject by:
a) navigating the distal tip of the cannula to a location near the intraorgan or extravascular site in the subject;
b) directing the distal tip toward a vessel wall in a general direction of the intraorgan or extravascular site;
c) advancing the distal tip such that the distal tip penetrates the vessel wall and reaches the intraorgan or extravascular site;
d) injecting the dose into the intraorgan or extravascular site; and
e) retracting the distal tip of the cannula from the intraorgan or extravascular site of the subject.

31. The method of claim 30, comprising repeating steps a) through e) with a second dose of the therapeutic.
